(19) United States
(12) Patent Application Publication
(10) Pub. No.: US 2006/0111423 A1
(43) Pub. Date: May 25, 2006

(54) Use of diindolylmethane-related indoles and growth factor receptor inhibitors for the treatment of human cytomegalovirus-associated disease

(76) Inventor: Michael A. Zeligs, Boulder, CO (US)

Correspondence Address: JONES DAY, 222 EAST 41ST ST, NEW YORK, NY, 10017, US

(21) Appl. No.: 11/260,543

(22) Filed: Oct. 26, 2005

Related U.S. Application Data
(60) Provisional application No. 60/622,333, filed on Oct. 26, 2004.

(51) Int. Cl. A61K 31/655; A61K 31/405; A61K 31/407

(52) U.S. Cl. 514/410; 514/414; 514/151

(57) ABSTRACT

The present invention includes compositions and methods for the treatment and prevention of conditions associated with Human Cytomegalovirus (HCMV) infection. HCMV-associated conditions include infections (active and latent), benign cell-proliferative conditions, pre-cancerous cell-proliferative conditions, and cancerous conditions. In particular, the present invention describes new therapeutic and preventative uses for 3,3′-diindolylmethane (DIM), or a DIM-related indole, in combination with an inhibitor of a membrane bound Growth Factor Receptor (GFR), to treat conditions associated with exposure to HCMV. In certain embodiments, the compositions of the invention can be used in combination with radiation therapy.

This application claims the benefit of U.S. Provisional Application No. 60/622,333, filed on Oct. 26, 2004, which is incorporated herein by reference in its entirety.

### 1. FIELD OF THE INVENTION

The present invention includes compositions and methods for the treatment and prevention of conditions associated with Human Cytomegalovirus (HCMV) infection. HCMV-associated conditions include infections (active and latent), benign cell-proliferative conditions, pre-cancerous cell-proliferative conditions, and cancerous conditions. In a particular embodiment, the present invention describes new therapeutic and preventative uses for 3,3′-diindolylmethane (DIM), or a DIM-related indole, in combination with an inhibitor of a membrane bound Growth Factor Receptor (GFR), to treat conditions associated with exposure to HCMV. In certain embodiments, the compositions of the invention can be used in combination with radiation therapy.

### 2. BACKGROUND OF THE INVENTION

2.1 The role of Human Cytomegalovirus (HCMV) and Growth Factor Receptors (GFR) in Disease

2.1.1 Biology of HCMV

HCMV is one of a diverse group of DNA viruses which have been implicated as initiators and contributors to cell-proliferative diseases, including certain types of cancer. HCMV is a distinct genera of the Betaherpesvirinae subfamily of DNA viruses, different genetically, structurally, and behaviorally from subfamily members of the Alphaherpesvirinae and Gammaherpesvirinae subfamilies of Human Herpes Viruses.

The activities of HCMV causing persistent, but unapparent infection, triggering unscheduled cell growth, and transforming cells into immortal variants implicates the virus in a broad spectrum of disease including infections, benign cell-proliferative disorders, and certain pre-cancerous and cancerous conditions. Once having gained entry into a permissive cell type, HCMV often becomes latent, i.e, a non-productive infection. Latent infection, which is lifelong, describes the state of HCMV viral DNA maintaining itself in cells without discernable abnormalities in cell structure or behavior. Latency refers to the presence of the viral genome in specific cells with production of latency-associated transcripts (LATs) from the viral genome, but no viral replication (Wagner and Hewlett, Basic Virology, Blackwell Scient Ltd, Malden Mass., 2004, pp. 38-39). Latency is contrasted to viral persistence which involves continual low level production of active virions. Persistent HCMV infection only occurs in certain tissues and cell types. Following primary infection with transient viremia, HCMV preferentially occupies the monocyte class of white blood cells and their precursors residing in bone marrow. HCMV is carried in a latent state within monocytes to tissue sites of inflammation, where differentiation of monocytes into macrophages triggers re-activation of HCMV and a persistent low level infection. HCMV infected monocytes may thus contribute to various, distinct chronic inflammatory conditions (Sissons et al., 2002, J Infect. 44:73-7). HCMV DNA has been identified in monocytes present in hypertrophic prostate tissue (Stapleton et al., 1996, J Urol. 156:542-5), at sites of chronic inflammation involving the intestines (regional enteritis [Crohn's Disease] and ulcerative colitis) (Hommes et al., 2004, Inflamm Bowel Dis. 10:245-50), in the lungs of patients with Idiopathic Pulmonary Fibrosis (Tang et al., 2003, J Clin Microbiol. 41:2633-40), in kidneys of patients with interstial nephritis (Platt et al., 1985, Kidney Int. 28:550-2), and in coronary artery atheromas following angioplasty (Radke et al., 2001, Coron Artery Dis. 12:1-6). HCMV specific DNA has been recently identified in pre-cancerous and cancerous prostate gland cells (Samanta et al., 2003, J Urol. 170:998-1002), in pre-cancerous and cancerous colon tissue (Harkins et al., 2002, Lancet 360:1557-63), and in non-melanoma skin cancer (Zafiropoulos et al., 2003, Cancer Lett 198:77-81). HCMV infects leukocytes, endothelial cells, connective tissue cells, and epithelial cells and is transmitted through milk, semen, urine, saliva, and cervical secretions. HCMV infection is acquired via the transplacental, perinatal and sexual routes and through blood transfusion and organ or bone marrow transplantation.

When symptomatic, primary HCMV infections in children and adults can cause polyneuritis, myelitis, and heterophile-negative mononucleosis syndrome, carditis and hepatitis. HCMV infection is especially a problem in individuals with weak immune systems, including newborns, infants, and those with inherited or aquired immune deficiency. HCMV is the most common cause of viral birth defects in congenitally infected babies, including congenital deafness and pneumonia. In immunocompromised individuals, HCMV can cause severe disseminated disease characterized by chorioretinitis, pneumonia, esophagitis, colitis, myelitis, meningitis, encephalitis, and hepatitis. In addition, HCMV causes severe retinitis in AIDS patients, which can lead to blindness. Latent HCMV infections can reactivate following blood transfusions, pregnancy, solid-organ or bone marrow transplantations, immunosuppressive therapy, or other viral infections.

Benign proliferative conditions associated with the presence of HCMV include inflammatory bowel disease (regional enteritis [Crohn's Disease] and ulcerative colitis) (Hommes et al., 2004, Inflamm Bowel Dis. 10:245-50), Idiopathic Pulmonary Fibrosis (IPF) (Yonemaru et al., 1997, Eur Respir J. 10:2040-5), renal fibrosis associated with interstitial nephritis (Platt et al., 1985, Kidney Int. 28:550-2), HCMV-associated macular degeneration (Miller et al., 2004, Am J Ophthalmol. 138:323-8), and certain forms of atherosclerosis (Chen et al., 2003, Mol Cell Biochem. 249:91-6). HCMV is associated with abnormal proliferation of endothelial and vascular smooth muscle cells in the arteries of transplanted organs. Transplant recipients with active HCMV infection often develop localized atherosclerosis following heart, lung, kidney and liver transplants often resulting in failure of the transplanted organ (Valantine, 2004, Am J Transplant. 4:169-77). Similarly, HCMV has been associated with intimal hyperplasia and arterial restenosis following arterial angioplasty and mechanical stenting of coronary arteries (Radke et al., 2001, Coron Artery Dis. 12:1-6).

2.1.2 Importance of Membrane Bound Growth Factor Receptors (GFR's)

Membrane bound Growth Factor Receptors (GFR's), also known as Receptor Tyrosine Kinases, are cell surface transmembrane proteins that, upon binding of extracellular growth factors, activate cytoplasmic signaling pathways. This results in a diverse array of cellular functions including cell differentiation, proliferation, migration and invasion, angiogenesis, and regulation of apoptosis (programmed cell death). The GFR's are enzymes that catalyze the transfer of a phosphate group from ATP to a tyrosine residue located on intracellular protein substrates. GFR's respond to various peptide growth factors in the extracellular space. The interaction of growth factors with GFR's is a necessary event in normal regulation of cell growth. However, under certain conditions these receptors can become deregulated resulting in uncontrolled cell proliferation, pre-cancerous proliferation of abnormal cells (dysplasia or intraepithelial neoplasia), and ultimately cancer (spreading neoplasia) (Tsatsanis et al., 2000, Int J Mol Med. 5:583-90). Among the important GFR tyrosine kinases are the members of the EGF receptor (EGFR) family. The EGFR family consists of four family members: EGFR (also known as erbB1 or HER1), erbB2 (HER2), erbB3 (HER3), and erbB4 (HER4). The EGFR family is activated by both EGF and Transforming Growth Factor Alpha (TGF-alpha). Overexpression of the HER2 receptor kinase has been associated with human breast, ovarian, colon, non small cell lung, and pancreatic cancer. Other GFR's include the Platelet Derived Growth Factor Receptor (PDGFR), the Vascular Endothelial Growth Factor Receptor (VEGFR), the Insulin-like Growth Factor Receptors (IGF-R and IGF-2R), Fibroblast Growth Factor Receptor (FGFR), Keratinocyte Growth Factor Receptor, and Hepatocyte Growth Factor Receptor (HGFR).

Overactive signaling through various GFR's is seen in a wide spectrum of cancer and is also associated with resistance to chemotherapeutic drugs and radiation therapy. Overexpression of the HER1 GFR is associated with a poor prognosis in ovarian, head and neck, esophageal, cervical, bladder, breast, colorectal, gastric, and endometrial cancer. HER2 overexpression is associated with poorer outcome in breast, ovary, prostate, lung, and bone cancer.

2.1.3 HCMV can Induce Activation of GFR's Analogous to that Seen with Various Cancers

Viruses have evolved molecular strategies of activating various GFR signaling pathways to cause a shift in cellular metabolism from a resting state to one associated with cell growth, DNA synthesis, and cell division. In many cases this activity involves inducing increased activity of the tyrosine kinases and inhibition of various triggers of programmed cell death (apotosis). Normally, apoptosis helps eliminate virally infected and pre-cancerous cells. Once internalized into a permissive cell type, HCMV produces virus specific proteins which inhibit the cellular process of apoptosis (Goldmacher et al., 1999, Proc Natl Acad Sci USA 96:12536-41). HCMV immediate-early (IE) proteins activate anti-apoptotic cellular growth and survival pathways including MAPK following infection (Rodems et al., 1998, J Virol. 72:9173-80). Oncoproteins from HCMV reduce levels of Retinoblastoma protein, a major apoptosis-promoting protein. Oncoproteins from HCMV activate the phosphatidylinositol 3-Kinase (PI3K)/Akt pathway resulting in anti-apoptotic cell signaling permissive for transformation to a pre-cancerous state. This causes infected cells to persist and undergo abnormal, unscheduled cell-division while harboring viral DNA (Yu et al., 2002, J Virol. 76:3731-8). More recently, HCMV has been shown to possess the specific attribute of gaining entry to the cell by physically interacting with HER1 (Compton, 2004, Trends Cell Biol. 14:5-8).

The connection of HCMV to cancer in which overexpression of GFR's occurs remains controversial since infection with the HCMV is known to result in downregulation of HER1, and a decrease in the cell surface expression of HER1 (Beutler et al., 2003, Am J Respir Cell Mol Biol. 28:86-94 and Fairley J, 2002, J Gen Virol 83:2803-2810).

2.1.3.1 Phosphatidylinositol 3-Kinase (PI3K) Activation Follows GFR Activation and Results in Signal Transduction Activating PKB/Akt

Activation of GFR's by extracellular growth factors results in transmembrane signaling and activation of Phosphatidylinositol 3-Kinase (PI3K) enzymes which generate a family of intracellular 3-phosphorylated lipid products. These 3-phosphoinositides serve as second messengers to recruit the major intracellular kinase, Protein Kinase B (PKB/Akt), to the plasma membrane and to alter its conformation. This permits selective phosphorylation and activation of PKB/Akt by phosphoinositide-dependent kinase-1 (PDK-1). Activated PKB/Akt is then released intracellularly to phosphorylate and regulate the function of many cellular proteins involved in processes that include metabolism, apoptosis, and cell proliferation. Recent evidence suggests that PKB/Akt becomes activated in response to diverse stimuli in addition to growth factors including, hormones, extracellular matrix components, and certain viral infections. In addition, PKB/Akt is frequently constitutively active in many types of human cancer. Constitutive PKB/Akt activation can occur due to amplification of PKB/Akt genes or as a result of mutations in components of the signalling pathway that activates PKB/Akt. Although the mechanisms have not yet been fully characterised, constitutive PKB/Akt signalling is believed to promote proliferation and increased cell survival and thereby contributes to cancer progression (Nicholson et al., 2002, Cell Signal. 14:381-95). Activation of Akt to its phosporylated form is associated with a poor prognosis in lung and prostate cancer.

2.2 Cruciferous Indoles

2.2.1 Natural Indole Compounds are Associated with Protection from Certain Cancers

Cruciferous vegetables contain a family of plant protective compounds called glucosinolates which give rise to active compounds with indole rings exemplified by indole-3-carbinol (I3C). The action of I3C in cell culture models has been associated with the promotion of apoptosis in a variety of cell types including prostate cancer cells (Chinni et al., 2001, Oncogene 20:2927-36). I3C's pro-apoptotic activity has also been associated with reduced activity of certain GFR cell signaling pathways, including diminished Akt phosphorylation. Treatment with I3C reduced the levels of HER1 (EGFR) cell membrane proteins in prostate cancer cells in culture (Chinni et al., 2002, Clin Cancer Res 8:1228-36). However, I3C has also been shown to be inactive when tested in cell culture models relevant to prostate cancer cell migration and metastasis (Nwankwo, 2002, Anticancer Res. 22:4129-35). Moreover, I3C's use is associated with a number of safety concerns due to its enzyme-inducing and reproductive-toxic actions (Dashwood, 1998, Chem Biol Interact. 110: 1-5; Gao et al., 2002, Toxicol Appl Pharmacol. 183:179-88).

Oral ingestion of I3C results in the gastric conversion of I3C into at least twenty acid condensation products, many of which are bioavailable, the most prevalent of which include CTR (cyclic trimer), HI-IM, DIM, ICZ and LTr-1 (Stresser et al., 1995, Drug Metabolism and Disposition 23:965-975). The fact that there are many non-DIM acid condensation products of I3C, produced in vivo at equal or greater levels as DIM, which can be responsible for I3C's activity, requires that biologic activities of individual condensation products like DIM need to be demonstrated directly.

2.2.2 Diindolylmethane (DIM) is an I3C Derivative Which Also Promotes Apoptosis

DIM is one of many products derived from I3C and also present in cruciferous plants. Once formed, DIM is stable in acid. In cell culture, isolated DIM has been shown to have apoptosis promoting effects in both estrogen-dependent and independent breast cancer cells (Hong et al., 2002, Biochem Pharmacol. 63:1085-97). In animals, orally administered DIM inhibits the growth of certain chemically induced forms of breast cancer (Chen et al., 1998, Carcinogenesis 19:1631-9). Recently, DIM has been shown to specifically induce apoptosis in HPV oncogene altered cervical cancer cell lines (Chen et al., 2001, J Nutr. 131:3294-302). This cell culture work demonstrated that DIM was more active than I3C in inducing markers of apotosis. Other non-DIM I3C condensation products were not tested. Further work has utilized DIM in the cell culture of prostate cancer cell lines demonstrating it to have anti-androgen activity similar to non-indole antiandrogen drugs (Le et al., 2003, J Biol Chem. 278:21136-45). In vivo studies in mice suggest that expected effective plasma levels are not easily achieved in humans (Anderton et al., 2004, Drug Metab Dispos. 32:632-8).

While shown to be an anti-androgen in prostate cancer cells, DIM has also been shown to be estrogenic in breast cancer cells (Riby et al., 2000, Biochem. Pharmacol. 60:167-177) and in rainbow trout, a model of carcinogenesis relevant to viral disease in humans (Shilling et al., 2001, Toxicology and Applied Pharmacology 170:191-200). Since estrogenic effects inhibit apoptosis, DIM may actually enhance estrogen related growth and cancer cell survival. Based on the conflicting results of DIM activity in cell culture studies, it is difficult to predict DIM's effects in vivo on cancerous processes including breast and prostate cancer. In addition, DIM has been shown to promote the production of Transforming Growth Factor Alpha (TGF-alpha) in cell culture (Leong et al., 2001, Carcinogenesis 22:1809-17). TGF-alpha is present at elevated levels in prostate cancer and promotes cell division in prostate cancer cell lines. On this basis, DIM is of uncertain value in treating prostate cancer. Finally, DIM has been shown to activate the Mitogen Activated Protein Kinase (MAPK) cell signaling pathway in cell culture. Activated MAPK is associated with cancer promotion, cancer cell survival (Leong et al., 2004, Mol Endocrinol. 18:291-302), and active HCMV infection (Johnson et al., 2000, J Virol. 74:1158-67). These properties of DIM suggest that DIM would not be useful for treating HCMV infections and HCMV-related cancer. Thus, the prior scientific literature teaches that DIM is not a likely acid condensation product of I3C which may be responsible for I3C's anti-viral activity and that DIM itself is not a likely candidate for anti-HCMV activity.

2.3 Uses of GFR Inhibitors in Cancer

Multiple approaches to inhibiting activity of the GFR's have been developed to treat cancer. These include development of GFR type-specific monoclonal antibodies and small molecule inhibitor drugs. Preliminary clinical uses of GFR inhibitors have focused on treatment of cancers which demonstrate increased expression of the HER membrane GFR's. These uses include Gefitinib (Iressa®, Astra Zeneca, UK) in combination with chemotherapeutic drugs (gemcitabine, cisplatin, carboplatin, and paclitaxel) in pancreatic, ovarian, and non-small-cell lung cancer (NSCLC) (Perez-Soler, 2004, Oncologist 9:58-67). Gefitinib (Iressa), specific for HER1 (EGFR), is the only drug of the new class of quinazoline GFR inhibitors, which has been approved as monotherapy for Non Small Cell Lung Cancer (NSCLC). Its use has been associated with limited responses in NSCLC and plagued by dose related side effects. Erlotinib (Tarceva, OSI) is in clinical trials testing as monotherapy for resistant ovarian cancer. Neither ovarian or lung cancers are HCMV-related. No attempt has been made to treat HCMV-related cancers with GFR inhibitor agents alone or in combination with established chemotherapy or radiation. So far the therapeutic response to Gefitinib, Erlotinib, and other GFR inhibitors in solid tumors has been disappointing. Only a minority of tumors have responded with delayed growth. Treatment with these GFR inhibitors has not resulted in persistent elimination of tumor mass. Problems with the use of the GFR inhibiting therapies alone include limited response rate and dose limiting side effects. Dose dependent side effects include acnieform rashes and diarrhea. Thus, current uses of GFR inhibitor drugs are limited to advanced cancers resistant to first-line chemotherapy or radiation therapy since the selectivity of GFR inhibitors for tumor tissue and long term safety of inhibiting GFR's in normal tissue are not known.

Proposals exist for the combined use of GFR inhibitors with other approved chemotherapeutics and with radiation therapy. These include combined use of multiple GFR inhibitors concurrently including Gefitinib (Iressa) and monoclonal antibodies such as Transtuaumab (Herceptin). No clinical trials of GFR inhibitor drugs or monoclonal antibodies have been reported in HCMV-related cancer (prostate, or colon).

2.4 Need for Better Therapy for HCMV-Related Disease

Individuals suffering from diseases caused by HCMV are currently treated with ganciclovir, valganciclovir, cidofovir, and phosphocarnet, which block the viral life cycle by inhibiting viral DNA replication. However the substantial toxicity of these drugs, treatment failures, and the emergence of drug-resistant strains of HCMV indicate that better antiviral compounds and treatments are needed.

Since current anti-virals in use against active HCMV-related infection or progression of latent HCMV-related processes are only active when there is ongoing replication of HCMV DNA, better therapies are needed which target both quiescent HCMV-altered cells in which in which there is little to no viral replication as well as active HCMV infections. Since the presence of HCMV within cells increases activity of PKB/Akt and MAPK, thereby maintaining latent infection, therapies with cell-penetrating small molecule drugs which inhibit these pathways would be expected to promote apoptosis in HCMV infected cells before mature progeny viruses can be produced. One approach would be to selectively induce apoptosis in HCMV altered cells. No prior attempt has been made to use GFR inhibitors to treat active or latent viral infections. No anti-HCMV uses of GFR inhibitors are described as therapeutic targets in GFR inhibitor literature or patents. Moreover, there is a low expectation that using GFR inhibitor agents will be clinically useful in HCMV-related conditions since reduced numbers of membrane-bound HER1(EGFR) receptors have been found following HCMV infection in vitro (Beutler et al., 2003, Am J Respir Cell Mol Biol. 28:86-94).

Apart from the use of certain DNA nucleoside anti-viral drugs which are marginally effective and frequently toxic, there are no available therapeutic agents for HCMV which target latent viral activity in monocytes or localized, persistent HCMV-infection. The limited effectiveness of current HCMV anti-viral drugs in active HCMV infection does not meet the need for treating or preventing HCMV infections in people with weak immune systems, such as immuno-compromised individuals and newborn infants. There is also a need to treat pregnant women having HCMV infections to prevent transmission to the offspring.

The current anti-viral drugs are of no benefit in latent HCMV infection and HCMV-associated pre-cancer and cancer. Since HCMV contributes to cell-proliferative diseases of cardiovascular, prostate glandular, gastrointestinal, and pulmonary tissues, and to a number of prevalent cancers which lack adequate therapy, effective modalities for controlling HCMV-associated disease need to be developed. There is a clear need for improved treatments for HCMV related infections, HCMV-associated cell-proliferative disorders and HCMV-associated cancers.

### 3. SUMMARY OF THE INVENTION

The present invention concerns new treatments for a range of Human Cytomegalovirus (HCMV) related disorders. The invention provides methods of use of Diindolylmethane-related indoles in combination with various GFR inhibitors in the prevention and treatment of HCMV-associated disease. Certain combinations are directed at early and late stages of HCMV viral infections and uses regarding HCMV-associated pre-cancers and cancers.

In certain embodiments of the invention, methods combine Diindolylmethane-related indoles with GFR inhibitors, including, but not limited to, monoclonal antibodies and small molecule GFR inhibiting drugs, to treat HCMV-related infections, benign cell-proliferative conditions, pre-cancerous and cancerous conditions. Benign cell proliferative conditions include post-transplant intimal hyperplasia, HCMV-related atherosclerosis, HCMV associated post-allograft organ transplant vasculopathy, neovascular age-related macular degeneration, inflammatory bowel disease, arterial restenosis following angioplasty, vascular graft associated intimal hyperplasia in renal failure, idiopathic pulmonary fibrosis, and chronic interstitial nephritis. In a preferred embodiment, the methods of the invention are used for the prevention and treatment of pre-cancerous conditions of uncontrolled or abnormal cell growth which result from deregulation of GFR's, including, but not limited to, neovascular age-related macular degeneration, abnormal growth of of prostatic glandular epithelium (Prostatic Intraepithelial Neoplasia [PIN]), and of colonic epithelium (Colonic Polyposis). In another preferred embodiment, these methods are used for the prevention and treatment of prostate cancer or colon cancer. Methods of treatment are described which improve responses to standard radiation therapy and chemotherapy when co-administered with one or more of small molecule GFR inhibiting drugs, GFR inhibiting Mab's, and Diindolylmethane-related indoles.

In another embodiment, the present invention relates to methods for treating or preventing a HCMV-related disorder comprising administering to a subject in need thereof a therapeutically effective amount of a DIM-related indole, alone or in combination with a GFR inhibitor.

In yet another embodiment, the present invention relates to methods for treating or preventing neovascular age-related macular degeneration comprising administering to a subject in need thereof a therapeutically effective amount of a DIM-related indole, alone or in combination with a GFR inhibitor.

In certain embodiments, these methods employ structurally-related, synthetically-derived, substituted diindolylmethane compounds. In a particular embodiment, the one or more DIM-related indoles of the invention are selected from the group consisting of 3,3′-diindolylmethane (DIM), hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolyl-3,3′-diindolylmethane, nitro-substituted imidazolyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

In a particular embodiment, the DIM-related indole and a GFR inhibitor are administered simultaneously. In another embodiment, the DIM-related indole and a GFR inhibitor are administered within a short time of one another, for example, 30 seconds, 1 minute, 5 minutes, 15 minutes, 30 minutes, 1 hour, 4 hours, 8 hours, 12 hours or 24 hours of one another.

In an additional embodiment, the combination of a DIM-related indole and a GFR inhibitor is administered in conjunction with differentiation promoting agents which help convert HCMV infected monocytes into therapy sensitive macrophages. Differentiation promoting agents include Vitamin-D, Vitamin-A (retinoids), and macrophage colony stimulating factors.

In a further embodiment, the combination of DIM-related indoles and GFR inhibitors are administered in conjunction with a radiation therapy regimen sufficient to treat a HCMV-related disease. In a preferred embodiment, topical ultraviolet light or site directed ionizing radiation (X-rays) is used. In another further embodiment, the combination of DIM-related indoles and GFR inhibitors are administered in conjunction with a photodynamic therapy regimen sufficient to treat a HCMV-related disease.

The invention further provides pharmaceutical compositions, for example, a pharmaceutical composition comprising a therapeutically effective amount of the combination of a diindolylmethane-related indole and a GFR inhibitor. In particular embodiments, the composition is formulated for oral or topical administration.

### 4. DETAILED DESCRIPTION OF THE INVENTION

The invention concerns methods and compositions of preventing and treating Human Cytomegalovirus (HCMV) infections and HCMV-related diseases utilizing Diindolylmethane (DIM)-related indoles in combination with GFR inhibitors. The combined activity of DIM-related indoles and GFR inhibitors describes a new mode of treatment of HCMV infections directed at promoting programmed cell death (apoptosis) of HCMV infected cells. The methods and compositions provide improved treatment for HCMV-related infections, benign cell-proliferative disorders, and for HCMV-associated cancers. Cancer-related treatments using DIM-related indoles and GFR inhibitors include the use of oral and parenteral preparations in conjunction with radiation therapy. Thus, in one embodiment, DIM-related indoles and GFR inhibitors used together serve to overcome radiation induced resistance to apoptosis and increase the therapeutic efficacy of radiation therapy.

In another embodiment, the methods of the invention are used for the prevention and treatment of pre-cancerous conditions of uncontrolled or abnormal cell growth which result from deregulation of GFR's, including, but not limited to, neovascular age-related macular degeneration, abnormal growth of of prostatic glandular epithelium (Prostatic Intraepithelial Neoplasia [PIN]), and of colonic epithelium (Colonic Polyposis).

In yet another embodiment, the methods of the invention are used for the prevention and treatment of neovascular age-related macular degeneration, regardless of whether an HCMV infection is present. As used herein, “neovascular age-related macular degeneration” and “neovascular macular degeneration” are used interchangeably. Neovascular macular degeneration, treated according to the methods of the invention, is also called “wet” or “exudative” macular degeneration and is associated with choroidal neovascularization.

The invention is based, in part, on expected synergism in using particular combinations of DIM-related indoles and GFR inhibitors with apoptosis promoting activity in HCMV infected cells. The combination of a DIM-related indole and a GFR inhibitor represents a new approach to HCMV infection and HCMV-associated disease. The unique aspect of the combined activity of compounds with different structures and mechanisms of action has to do with selective elimination of HCMV disrupted cells with little toxicity to uninvolved bystander cells. Combined use permits lower dose use of GFR inhibitors, reducing dose-related side effects of these drugs. In certain embodiments, the compositions of the invention can be used with differentiation promoting agents such as Vitamin-D derivatives, retinoid derivatives, and macrophage colony stimulating factors. In certain embodiments, the methods of the invention involve the use of DIM-related indoles, a GFR inhibitor and radiation therapy. The combination of a DIM-related indole and a GFR inhibitor is believed to induce promotion of apoptosis resulting in the selective elimination of abnormal cells, and causes resolution of HCMV-related lesions of epithelial surfaces, endothelial surfaces, and epithelial glands. Combinations of existing small molecule GFR inhibiting drugs with DIM-related indoles are used to increase the effectiveness and reduce the toxicity of existing cancer treatments for HCMV-associated cancers.

Without being bound by any theory, the methods and compositions of the invention are believed to diminish overactivity and inhibit cell signaling pathways downstream of HER receptors, thereby promoting apoptosis in abnormal cells. The methods and compositions of the invention are therefore useful for treating certain disease states, including certain HCMV-related disease states, and some types of cancer that result, in part, from unregulated activity of these receptor-driven signaling pathways.

HCMV infection of cells initiates cell-growth and cell-survival mechanisms uniquely attributed to the action of HCMV-specific proteins. One of the primary abnormalities in cellular activity is a change in expression and activity of the membrane bound Growth Factor Receptors (GFR) family of signaling proteins (Johnson et al., 2001, J Virol. 75:6022-32). In certain embodiments, the present invention provides new combined therapy in HCMV-related pre-cancerous conditions of the prostate gland and colonic epithelium. In one embodiment, DIM-related indoles and GFR inhibitors are used in fully developed cancer of the prostate, colon and skin. In another embodiment, DIM-related indoles and GFR inhibitors are used in conjunction with existing chemotherapy and radiation therapy to better treat these HCMV-related cancers.

HCMV provides an activation signal for cell survival through the PI3K-Akt kinases which inhibits cellular apoptosis. Specific oncoproteins from HCMV promote phosphorylated Akt and inhibit apoptosis. Normally, activation of Akt occurs through occupation of cell surface Growth Factor Receptors (GFR's). Principal GFR's include the Epidermal Growth Factor Receptor (EGFR [HER1]), related receptors in the HER family of receptors (HER2-4), the Platelet Derived Growth Factor Receptors (PDGFR), and the Vascular Endothelial Growth Factor Receptors (VEGFR). In HCMV-related pre-cancer and cancer, the presence of viral oncoproteins within cells promotes aberrant activation of GFR's through interaction with the internal domain of the GFR protein projecting within the plasma membrane, making GFR activation independent from stimulation of growth factors acting external to the cell. This results in maintenance of viral DNA within cells without viral replication (latent infection) or active viral replication with release of mature viral particles further infecting surrounding cells (active infection).

Without being bound by any theory, the present invention employs DIM-related indoles and GFR inhibitors to inhibit the HCMV-associated activation of PI3K-Akt and MAPK and selectively induce apoptosis in actively and latently HCMV-infected cells, thereby reducing latently infected cells and eliminating virally damaged cells. This activity can prevent low-level persistent infection and active infection, and treat HCMV-associated pre-cancerous and cancerous cell-proliferative conditions. Prophylactic uses of DIM-related indoles with GFR inhibitors can prevent primary infection or re-infection with HCMV. Selective inhibition of overactive survival and growth signals in HCMV-infected cells in the present invention can provide effective therapy, causing virally altered cells to be eliminated by triggering programmed cell death (apoptosis). The present invention provides new methods for combined therapy with inhibitors of various GFR's and DIM-related indoles to selectively induce apoptosis and treat certain benign and malignant diseases. These disorders are associated with abnormal cellular survival and growth related to exposure to HCMV.

In one embodiment, the present invention relates to methods for treating or preventing benign and malignant cell-proliferative disorders using DIM and DIM-related indoles in combination with GFR inhibiting compounds. This combined use provides for more effective control of cell-proliferative disorders, including HCMV-related cell-proliferative disorders, than use of either compound alone, facilitating more successful reversal of benign proliferative disorders including post transplant arterial stenosis, neovascular age-related macular degeneration, idiopathic pulmonary fibrosis (IPF), interstitial nephritis, inflammatory bowel disease (IBD), and precancerous prostatic intraepithelial neoplasia (PIN). Combined use permits lower, less toxic doses than with higher dose use of either DIM-related indole or GFR inhibitor alone. The methods provide for more complete therapeutic control of certain malignant conditions, especially of epithelial origin, which may involve exposure to HCMV. These include cancer of prostate, cancer of the colon, and non-melanoma skin cancer. Further combination with the third treatment modality of radiation therapy increases the effectiveness of combined GFR, DIM-related indole, chemotherapy and radiation therapy in advanced cases of these malignancies.

4.1 Diindolylmethane-Related Indoles

The DIM-related indoles or DIM compounds useful in the methods and compositions of the invention include DIM (3,3′-diindolylmethane) and the related linear DIM trimer (2-(indol-3-ylmethyl)-3,3′-diindolylmethane [also written: 2 (Indol-3-ylmethyl)-indol-3-yl]indol-3-ylmethane] (LTR). As used herein, “DIM-related compound”, “DIM-related indole”, and “DIM derivative” are used interchangeably, and refer to both natural metabolites and analogs of DIM, and also to “structurally-related, synthetically-derived, substituted diindolylmethane compounds” and “synthetic derivatives of DIM”, such as those disclosed herein and known in the art. As used herein, “cruciferous-related indoles” encompasses the terms “DIM-related compound”, “DIM-related indole”, and “DIM derivative”. One of ordinary skill in the art will recognize that in any of the pharmaceutical compositions or methods of the invention where DIM is used, a DIM-related compound, including a structurally-related, synthetically-derived, substituted diindolylmethane compound or synthetic derivative of DIM, can be used.

The chemical structure of a DIM is as follows (where each of the R groups is H):

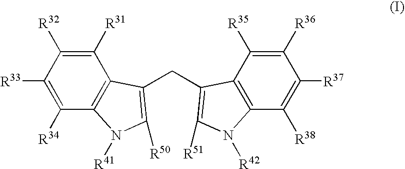

The chemical structure of LTR is as follows (where each of the R groups is H):

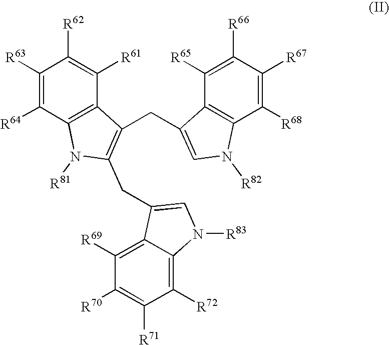

In certain embodiments, an active hydroxylated or methyoxylated metabolite of DIM, i.e., a compound of formula I, wherein R³², R³³, R³⁶, and R³⁷ are substituents independently selected from the group consisting of hydrogen, hydroxyl, and methoxy, and R³¹, R³⁴, R³⁵, R³⁸, R⁴¹, R⁴², R⁵⁰, and R⁵¹ are hydrogen, is utilized.

In certain embodiments, an active hydroxylated or methyoxylated metabolite of LTR, i.e., a compound of formula II, wherein R⁶², R⁶³, R⁶⁶, R⁶⁷, R⁷⁰, and R₇₁ are substituents independently selected from the group consisting of hydrogen, hydroxyl, and methoxy, and R⁶¹, R⁶⁴, R⁶⁵, R⁶⁸, R⁶⁹, R⁷², R⁸¹, R⁸², and R⁸³ are hydrogen, is utilized.

In an alternative embodiment, active DIM derivatives with R₃₂ and R₃₆ substituents made up of ethoxycarbonyl groups, and R₅₀, R₅₁ are either hydrogen or methyl, are utilized. In another embodiment, active substituted DIM derivatives including methylated and chlorinated compounds, exemplified by those that include 5,5′-dimethylDIM (5-Me-DIM), 2,2′-dimethylDIM (2-Me-DIM), and 5,5′-dichloroDIM (5-Cl-DIM) are described in U.S. Patent Application Publication No. 20020115708 by Safe, published Aug. 22, 2002, incorporated herein by reference in its entirety, are utilized in the present invention. In another embodiment, active DIM derivatives include imidazolelyl-3,3′-diindolylmethane, including nitro substituted imidazolelyl-3,3′-diindolylmethanes, and additional DIM-related compounds described in U.S. Patent Application Publication No. 2004/0043965 by Jong, Ling, published Mar. 4, 2004, incorporated herein by reference in its entirety, are utilized. In yet another embodiment, active DIM derivatives include substituted DIM derivatives described in U.S. Patent Application Publication No. 2005/0058600, by Bjeldanes LF, Le HT, and Firestone GL.

In certain embodiments, a DIM related compound has formula (III):

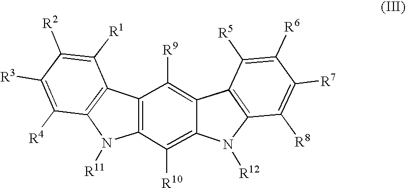

wherein:

R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms; and

R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,

with the provisos that: at least one of R¹, R², R³, R⁴, R⁵, R⁶, R⁷, R⁸, R⁹, R¹⁰, R¹¹ and R¹² is other than hydrogen; and when R¹, R², R³, R⁴, R⁵, R⁶, R⁷, and R⁸ are selected from hydrogen, halo, alkyl and alkoxy, then R¹¹ and R¹² are other than hydrogen and alkyl.

A preferred embodiment includes the use of 2,10-dicarbethoxy-6-methoxy-5,7-dihydro-indolo-[2,3-b]carbazole (SRI13668 (SRI Inc., Menlo Park, Calif.)). Additional preferred embodiments include the use of 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole (SRI Inc., Menlo Park, Calif.).

In another embodiment, a DIM related compound has formula (IV):

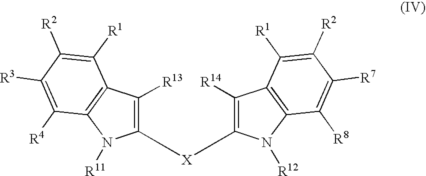

wherein:

R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;

R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl;

R¹³ and R¹⁴ are defined as for R¹, R², R³, R⁴, R⁵, R⁶, R⁷, and R⁸, with the proviso that at least one of R¹³ and R¹⁴ is other than hydrogen; and

X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹².

A preferred embodiment includes the use of 2,6-dicarbethoxy-3,3′-dimethyl-13,14-diindolylmethane (SRI Inc., Menlo Park, Calif.).

In another embodiment, a DIM related compounds has formula (V):

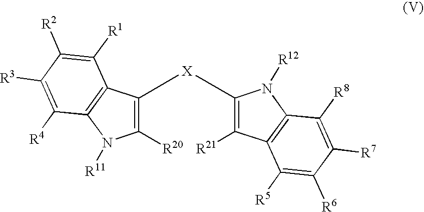

wherein:

R¹, R², R³, R⁴, R⁵, R⁶, R⁷, R⁸, R¹¹, R¹², and X are defined as for compounds of formula (III); and

R²⁰ and R²¹ are defined as for R¹, R², R³, R⁴, R⁵, R⁶, R⁷, and R⁸.

In yet another embodiment, the DIM-related indole is an indole-3-carbinol tetrameric derivative (Brandi et al., 2003, Cancer Res. 63:4028-4036).

4.2 Growth Factor Receptor Inhibitors

The GFR inhibitors of use in the present invention include, but are not limited to, small molecule drugs which inhibit one or more GFR, monoclonal antibodies inactivating GFR's, and antisense DNA or RNA inactivating GFR DNA or RNA delivered to cell using gene therapy. GFR's which may be inhibited include any GFR known in the art. See, e.g., Rajkumar, 2001, Current Science 81:535-541.

Small molecular GFR inhibitors suitable for use in the invention include the EGFR inhibitors, Gefitinib (N-(3-chloro-4-fluorophenyl)-7-methoxy-6-(3-morpholinopropoxy)quinazolin-4-amine, Iressa®, AstraZeneca, UK) and related compounds (see European Patent Application No. 0566226; International Patent Applications WO 96/33980 and WO 97/30034; Woodburn et al., 1997, Proc. Amer. Assoc. Cancer Research 38:633; and Woodburn et al., 1999, Pharmacol. Ther. 82, 241-250), Erlotinib (N-(3-ethynylphenyl)-6,7-bis(2-methoxyethoxy)quinazolin-4-yl]-(3-ethynylphenyl)amine, Tarceva®, OSI Pharmaceuticals) and related compounds (see International Patent Applications WO 96/30347 and WO 99/55683), CI 1033 (6-acrylamido-N-(3-chloro-4-fluorophenyl)-7-(3-morpholinopropoxy)quinazoli-n-4-amine, Pfizer) and related compounds (see International Patent Applications WO 97/38983 and WO 00/31048, and Smaill et al., J. Med. Chem., 1999, 42:1803-1815), PKI 166 (4-[(1R)-1-phenylethylamino]-6-(4-hydroxyphenyl)-7H-pyrrolo[2,3-d-]pyrimidine, Novartis Pharma, AG [Basel]) and related compounds (see International Patent Application WO 97/02266), Baicalein (Evers et al. Antiviral Res. 2005 Sep. 24, epublication) and, Baicalin (LKB Laboratories, Inc. St. Paul, Minn.).

Other membrane-bound GFR inhibiting drugs pertinent to the present invention include small molecule drugs and monoclonal antibodies which inhibit the activity of the platelet-derived growth factors (PDGF's) and the Vascular Endothelial Growth Factors (VEGF's). For example, PTK787/ZK 222584 (PTK787) (Vatalanib, Schering/Novartis) is an orally active anilino-phthalazine designed to bind specifically to the tyrosine kinase domain of both VEGFR types and inhibit angiogenesis. SU 10944 (3-[5-methyl-2-(2-oxo-1,2-dihydro-indol-3-ylidenemethyl)-1H-pyrrol-3-yl]-proprionic acid is an orally active pyrrole indolinone and potent inhibitor of VEGFR-2 also useful in the present invention. Macugen (pegaptanib sodium injection [Pfizer]), an injectable DNA fragment, or aptamer, specifically inhibiting VEGF which can also used in combination with DIM-related indoles according to the present invention. The GFR inhibitor imatinib mesylate (formerly ST1571, Gleevec, Novartis Pharmaceuticals Corp, East Hanover, N.J.) is a potent inhibitor of PDGFR kinase. Differentiating promoting agents can also be used in conjunction with GFR inhibitors. Differentiating promoting agents useful in the present invention include, but are not limited to, Vitamin D3, calcitriol (Rocaltrol, Roche Labs, Nutley, N.J.), Vitamin A, a retinoid derivative, such as isotretinoin (Acutane, Roche Labs, Nutley, N.J.), and granulocyte-macrophage colony stimulating factors such as sargramostin (Leukine, Berlex Labs).

Other examples of GFR inhibitors include, but are not limited to, resveratrol (Stewart et al., 2004, Invest. New Drugs 22:107-117) and epigallocatechin-3-gallate (Sah et al., 2004, J. Biol. Chem. 279:12755-12762).

The specific GFR's to be inhibited relate to those overactivating the PKB/Akt signaling pathway and include the HER family of GFR's, PDGFR, and VEGFR. Representative specific small molecule drugs useful in the present invention, presented in relation to the GFR inhibited are summarized in Table 1.

TABLE 1
Orally active and injectable GRF Inhibitor Drugs for Use with DIM-
Related Indoles:

| | | | HER | | | | | | |
|---|---|---|---|---|---|---|---|---|---|
| | | | EGFR | | | | VEGFR | | |
| Drug | Manufacturer | Drug Class | I | II | IV | IV | 1 | 2 | PDGF |
| ZD1839 | AstraZeneca | Small Head Group | X | | | | | | |
| Gefitinib | | Quinazoline | | | | | | | |
| (Iressa) | | (reversible) | | | | | | | |
| ZD6474 | AstraZeneca | | X | X | | | | | |
| OSI-774 | OSI//Roche/ | Small Head Group | X | | | | | | |
| Erlotinib | Genentech | Quinazoline | | | | | | | |
| (Tarceva) | | (reversible) | | | | | | | |
| Lapatinib | GlaxoSmithKline | Large Head Group | X | X | | | | | |
| GW-572016 | | Quinazoline | | | | | | | |
| GW-2016 | GlaxoSmithKline | | X | X | | | | | |
| STI-571 | Novartis | | X | | | | | | X |
| Imatinib | | | | | | | | | |
| Myesylate | | | | | | | | | |
| (Gleevec) | | | | | | | | | |
| EKB-569 | Wyeth | (irreversible) | X | X | | | | | |
| CI-1033 | Pfizer | 4-anilinoquinazoline | X | X | X | X | | | |
| (PD183805) | | (irreversible) | | | | | | | |
| Cancertinib | | | | | | | | | |
| SU5416 | Sugen | indolin-2-ketone | | | | | | X | |
| Semaxanib | Pharma/Pfizer | | | | | | | | |
| SU11248 | Sugen | indolin-2-one | | | | | | X | X |
| | Pharma/Pfizer | | | | | | | | |
| SU6669 | Sugen Pharma | | X | | | | | | |
| SU10944 | Sugen Pharma | pyrrole indolinone | | | | | | X | |
| Vatalanib | Novartis/Schering | anilino- | X | X | X | X | X | X | |
| PTK787 | | phthalazines | | | | | | | |
| (ZK222584) | | | | | | | | | |
| PKI-166 | Novartis | Pyrrolopyrimidines | X | X | | | X | | |
| | | (reversible) | | | | | | | |
| CEP-7055 | Sanofi-Synthelab | Dimethylglycene | | | | X | X | | |
| Pegaptanib | Pfizer/Eyetech | Aptamer | | | | | X | | |
| (Maucagen) | | | | | | | | | |

Representative specific GFR inhibiting monoclonal antibodies useful in the present invention presented in relation to the GFR inhibited, include those that appear in Table 2.

TABLE 2

| | Manu- | | | | |
|---|---|---|---|---|---|
| Drug | facturer | Class | HER | VEGFR | PDGF |
| Cetuximab | ImClone/ | Mouse/ | X | | |
| (Erbitux) | B-MS | human mAb | | | |
| Trastuzumab | Genentech/ | mAb | X | | |
| Herceptin | DNA | | | | |
| MDX-210 | Medarex | mAb | X | | |
| ABX-EGF | Abgenix/ | mAb | X | | |
| | Immunex | | | | |
| TheraCIM | YM | mAb | EGFR | | |
| Panitumumab | AbBenix | mAb | EGFR | | |
| EMD-72000 | Merck | mAb | EGFR | | |
| bevacizumab | DNA/ | mAb | | X | |
| Avastin | Hoffman | | | | |
| Ranibizumab | DNA/ | mAb | | X | |
| (Lucentis) | Novartis | | | | |

4.3 Preventation and Treatment of Human Cytomegalovirus (HCMV) Associated Conditions and Neovascular Age-Related Macular Degeneration

The present invention provides for the prevention and therapy of HCMV associated conditions which include infections, benign cell proliferative disorders, pre-cancerous conditions, and cancerous conditions. Currently, this spectrum of HCMV-related disease lacks adequate and effective therapy due to failure of previous approaches to selectively eliminate HCMV-altered cells and target the anti-apoptotic cell signaling pathways activated by HCMV and its virus specific proteins.

HCMV exhibits unique characteristics in its ability to maintain an inactive, quiescent carrier state in infected cells (latent infection). In individuals with prior exposure to HCMV, latent infection can persist in monocytes, a subset of white blood cells (leukocytes) which travel to sites of inflammation, exit the circulation and differentiate into macrophages (Sissons et al., 2002, J Infect. 44:73-7). This property of HCMV causes disease from newly activated HCMV in discrete locations and tissues initiated by non-infectious sources of inflammation as well as inflammation resulting from non-HCMV pathogens. The methods of treatment of the present invention thus include the treatment of benign HCMV-associated cell proliferative conditions including cases of prostate gland hypertrophy and prostatitis, post solid organ transplantation vasculopathy, post coronary angioplasty restenosis, coronary artery disease with HCMV seropositivity accompanied by stable or unstable angina, vascular graft associated intimal hyperplasia, neovascular age-related macular degeneration, idiopathic pulmonary fibrosis, renal fibrosis, inflammatory bowel disease (Crohn's Disease and ulcerative colitis), and colonic polyposis. For example, elderly men or women, with early visual loss and physical examination consistent with neovascular age-related macular degeneration, are candidates for DIM-related indole therapy.

The present invention also provides for the prevention and therapy of neovascular age-related macular degeneration, both HCMV-associated and non-HCMV-associated.

4.3.1 Methods of Prophylaxis

Protecting against a primary HCMV infection in a seronegative individual or protecting against re-activation of latent HCMV following seroconversion involves the preemptive or chemopreventive use of DIM-related indoles without a GFR inhibitor. Pregnant, immunocompetent women who serocovert becoming positive for IgM and/or IgG antibodies against HCMV are treated with DIM-related indoles for the duration of pregnancy and lactation. Women found to be HCMV seropositive prior to pregnancy are treated with DIM-related indoles prior to giving birth and during lactation, e.g., to prevent transmission to the fetus or newborn. Typically DIM-related indoles are used for chemoprevention by immuocompromised individuals with acquired HIV infection or AIDS, inherited immune dysfunction, or drug induced immune dysfunction following organ transplantation, following treatment of autoimmune disease, or following chemo/radiation treatment of cancer. The use of a GFR inhibitor in these patient populations would generally not be worth the risk of side effects associated with GFR inhibitors. Suppression of immune function results in activation of latent infection. In healthy individuals anti-HCMV cytotoxic T-cells continually attack HCMV infected cells that are producing virus. This controls infection and limits HCMV to localized sites where low level persistent infection is the result of continually arriving HCMV infected monocytes. So, by using DIM prophylactically apoptosis would be promoted in HCMV-infected cells as soon as active viral replication began as characterized by increased activity of PI3K and Akt.

Alternatively, DIM is used for HCMV prophylaxis in conjunction with differentiation enhancing agents like Vitamin-D, Vitamin-A, and macrophage colony stimulators which stimulate the final differentiation of monocytes into macrophages. Together with DIM an increased rate of conversion of monocytes into macrophages would increase the number of HCMV-infected macrophages that are vulnerable to DIM induced apoptosis.

4.3.2 Active Infections

Active HCMV-associated infections include intra-uterine infection, HCMV associated diabetes, HCMV associated CNS infection, HCMV associated polyneuritis, HCMV associated myelitis, and heterophile-negative mononucleosis syndrome, HCMV associated carditis, HCMV associated hepatitis, HCMV associated inflammatory bowel disease. Low level, persistent active infection is also seen in sexually transmitted HCMV. The presence of HCMV in semen and cervical secretions results in transmission between partners and chronic active infection of macrophages present in semen and uterine mucous. Replication and release of HCMV particles from macrophages in semen and seminal vessicles leads to chronic exposure and infection of prostate gland cells to HCMV virus. In immunocompromised individuals, the methods of the present invention provide for treatment of HCMV associated retinitis, chorioretinitis, pneumonia, esophagitis, colits, meningitis, and encephalitis. Treatment of the above conditions utilize DIM-related indoles administered alone and in combination with currently available HCMV anti-viral drugs which include ganciclovir, valganciclovir, cidofovir, and phosphocamet. The use of a combination of a DIM-related indole and a GFR inhibitor is generally not warranted in these cases, but may be used in severe cases.

4.3.3 Pre-Cancerous Conditions

The HCMV activated anti-apoptotic pathways of cell survival and proliferation, particularly activation of the GRF-asssociated PKB/Akt pathway, results in upregulation of sex steroid receptors including the androgen and estrogen receptors (see Ghosh et al, 2003, Curr Drug Metab. 4:487-96). Akt activation due to HCMV release from infected macrophages in inflamed epithelial tissue results in sex steroid related, cell proliferative, pre-cancerous conditions involving the prostate gland, colonic epithelium, and skin. Specifically, in certain embodiments, the methods of the present invention provide for treatment of precancerous prostatic intraepithelial neoplasia (PIN), and acquired colonic polyposis using a DIM related indole. In a preferred embodiment, a combination of a DIM-related indole and a GFR inhibitor is used.

4.3.4 HCMV-Associated Cancers

Active or latent HCMV infection also contributes to cancerous conditions where HCMV has activated cell survival signaling pathways. The methods of the present invention provide a new means to induce apoptosis in HCMV-associated cancer. HCMV-associated cancers include prostate cancer, colon cancer, malignant glioma, Eptein-Barr virus negative Hodgkin's disease, and non-melanoma skin cancer. In a preferred embodiment, the cancer is prostate cancer. The HCMV-associated cancerous conditions best treated by combinations of DIM-related indoles and GFR inhibitors are further identified by positive histochemical staining of tumor biopsy specimens for phosphorylated Akt utilizing monoclonal antibodies (Cell Signalling Technologies, Inc., Beverly, Mass.). Identification of HCMV-related tumors associated with elevations of Phospo Akt based on positive staining of tumor biopsies is accomplished as described in pending U.S. patent application Publication No. 20030190689. Thus, in certain embodiments, the methods of the present invention provide for treatment of prostate cancer and colon cancer using a DIM related indole. In a preferred embodiment, a combination of a DIM-related indole and a GFR inhibitor is used.

4.3.5 Neovascular Age-Related Macular Degeneration

The present invention also provides for the prevention and therapy of neovascular age-related macular degeneration, both HCMV-associated and non-HCMV-associated. While most neovascular age-related macular degeneration is believed to be due to HCMV infection, the methods of the invention encompass the prevention and treatment of the condition whether or not HCMV is detected or tested for.

4.4 Prevention and Treatment Parameters Using a DIM-Related Indole or Combined Use of DIM-related Indoles and GFR's

The invention provides for five (5) categories of treatment using DIM-related indoles and DIM-related indoles with GFR inhibitors: (I) chemoprevention of latent or primary HCMV infection with DIM-related indoles, (II) treatment of active HCMV infections with DIM-related indoles, with or without GFR inhibitors, and established HCMV anti-viral drugs, (III) treatment of HCMV-related benign cell-proliferative conditions with a combination of DIM-related indoles and GFR inhibitors (IV) treatment of HCMV-related pre-cancer with a combination of DIM-related indoles and GFR inhibitors, (V) treatment of HCMV-related cancer with DIM-related indoles and GRF inhibitor and optionally, radio-chemotherapy. In categories II-V, treatment includes the combination of DIM-related indole and GFR inhibitors with or without additional modalities of treatment.

Based on the category of treatment, in embodiments using a combination of a DIM-related indole and a GFR inhibitor, the DIM-related indole and GFR inhibitor can be used in 3 defined dose ranges. These dose ranges include Minimal Effective Dose (MED), Average Tolerated Dose (ATD), and Maximal Tolerated Dose (MTD). The MED relates to the lowest dosage range where biologic and metabolic effects from DIM-related indoles and GFR inhibitors are seen. The ATD is the dose range higher than the MED where consistent biologic effects are seen, but where side effects are rare. The MTD is the dose range higher than the ATD where side effects are often seen but are tolerable during the treatment protocol.

The following are general descriptions of DIM-related indole and combined DIM-related indole and GFR inhibitor therapy according to category of treatment.

I. Therapy of Primary and Latent HCMV Infections, Including Prophylactic Uses and Therapeutic Uses for Primary and Latent HCMV Infections

DIM-related indoles, with or without HCMV anti-viral agents, can be used to treat latent infection in newly converted seropositive individuals or seropositive individuals who are at risk for activation of HCMV. This applies to prospective solid-organ transplant recipients or seronegative recipients of donated organs from seropositive organ donors. It also applies to pregnant women who seroconvert during pregnancy or who are known to be HCMV seropositive and begin treatment prior to giving birth and during lactation in order to prevent perinatal transmission of HCMV to their newborn. Treatment with DIM-related indoles is also used by seropositive individuals wishing to prevent activation of latent HCMV who have pre-existing conditions including, but not limited to, HIV infection, AIDS, other aquired immunodeficiency, prostate gland hypertrophy, chronic prostatitis, presence of a transplanted solid organ, coronary artery disease accompanied by stable or unstable angina, presence of a vascular graft, and neovascular age-related macular degeneration. DIM-related indoles can also be taken prophylactically by sexually active seronegative individuals wishing to prevent sexually acquired HCMV primary infection. The therapy described in this section also apply to the prevention and treatment of neovascular age-related macular degeneration regardless of whether an HCMV infection is present.

Prophylactic treatment of HCMV infections and HCMV-related pre-cancerous conditions with DIM and Resveratrol are also provided. The combination of DIM-related indole and Resveratrol can be used by sexually active women and men to prevent men and women to reduce spread of HCMV from previously exposed individuals to un-exposed individuals. Typically a daily oral dose of 50-250 mg/day of DIM-related indole in a suitable formulation is taken along with a daily oral dose of 25-1000 mg of reseveratrol or resveratrol-related stillbene in a suitable formulation. Alternatively, the DIM-related indole and resveratrol can be used prophylactically in the form of a vaginal or rectal suppository.

II. Therapy of Active HCMV Infections

Active HCMV infections, as exemplified by AIDS associated retinitis, chorioretinitis, pneumonia, esophagitis, colits, myelitis, meningitis, encephalitis, and hepatitis, are treated with DIM-related indoles or a combination of a DIM-related indole and a GFR inhibitor. Combined DIM-related indole/GFR inhibitor therapy can be used with standard HCMV anti-viral drugs. The uses of the antiviral drugs are well known and specified in De Clerq (2004, J of Clinical Virology 30:115-133). In a preferred embodiment, DIM-related indoles and GFR inhibitors are used at higher doses than either alone with or without established HCMV anti-viral compounds.

For vision or life threatening conditions, DIM-related indoles would be administered with intravenous gancyclovir or to oral vancyclovir. As the clinical condition requires, an appropriate GFR inhibitor is further utilized.

In other embodiments, a DIM-related indole is used in combination with resveratrol. For example, a daily oral dose of 150-500 mg/day of DIM-related indole in suitable formulation is taken along with a daily oral dose of 25-1000 mg of reseveratrol or resveratrol-related stillbene in a suitable formulation. Alternatively, the DIM-related indole, gancyclovir or vancyclovir, and optionally resveratrol, can be used in combination with a GFR inhibitor. Typically, Gefitinib at 25-350 mg/day is added in serious cases. Following clinical improvement the DIM-related indole, with or without Resveratrol, is continued with the Gefitinib lower in its dose range. With stable clinical improvement the DIM-related indole with or without Resveratrol is continued.

III. Therapy of HCMV-Associated Benign Proliferative Disorders

HCMV-associated benign proliferative disorders include HCMV-related atherosclerosis (arterial restenosis following angioplasty and vascular graft associated intimal hyperplasia), progressive HCMV associated post-allograft organ transplant vasculopathy, progressive HCMV associated neovascular macular degeneration, active inflammatory bowel disease (Crohn's Disease), and idiopathic pulmonary fibrosis. For treatment of HCMV-related benign cell-proliferative conditions which include Idiopathic Pulmonary Fibrosis (IPF); neovascular macular degeneration, and post organ transplant vasculopathy the combined use of DIM-related indole and GFR inhibitor is undertaken at moderate to high doses. Since progressive HCMV-related proliferative disorders are serious life or vision threatening conditions, combined therapy with DIM-related indoles and GFR inhibitor drugs is generally undertaken at the highest tolerated dose of both agents for 1-3 months. Treatment of these vision or life threatening disorders supports the highest tolerated combined doses. Typically a daily oral dose of 300-750 mg/day of DIM-related indole in suitable formulation is taken along with a daily oral dose of 25-1000 mg of Gefitinib. Biopsy proven cases of IPF are treated for 3-6 months utilizing 300-750 mg/day of DIM-related indole in suitable formulation taken along with a daily oral dose of 25-1000 mg of Gefitinib.

In a related embodiment for neovascular age-related macular degeneration or transplant vasculopathy, treatment included formulated DIM administered at 150-750 mg twice daily and Gefitinib 25-750 mg once daily for 3-6 months. Optimally the doses would include formulated DIM 250 mg twice daily and Gefitinib 250 mg once daily for 4-6 months. Alternatively for neovascular age-related macular degeneration, DIM, administered 150-300 mg twice daily orally, is used in conjunction with a 0.3-3 mg intra-vitreal injection of Pegaptanib (Maucagen [Pfizer]), an anti-VEGF aptamer, given every 6 weeks. Optionally, bevacizumab (Avastin) (1.0 mg) can be used in place of the Pegaptanib. Alternatively, PTK787 [ZK222584] (Schering/Novartis) can be given orally at 750-1200 mg/day can be used in place of the intra-vitreal Pegaptanib or bevacizumab, used for a period of 3-6 months.

In another embodiment, neovascular age-related macular degeneration is treated a DIM-related indole, alone or in combination with a GFR inhibitor, in conjunction with Photodynamic Therapy (PDT). Preferably, the PDT utilizes ultraviolet light (UVA, UVB, UVC) and/or light emitted from a medical laser designed for ophthalmic use. Typically, the DIM-related indole, with or without an orally active GFR inhibitor drug, is given orally 2-6 hours before the ultraviolet or laser light treatment. Examples of techniques for utilizing PDT are provided in the medical literature (Wormald et al., 2005, Photodynamic therapy for neovascular age-related macular degeneration. Cochrane Database Syst Rev. 4:CD002030).

Doses and duration of therapy are adjusted based on the clinical condition and use of additional condition specific drug therapy. Alternatively, DIM can be administered in an ophthalmic suspension in place of, or in addition to, oral use. For less active benign proliferative disorders, DIM-related indoles can be used at lower doses in conjunction an oral HCMV antiviral agent (e.g., gancyclovir or vancyclovir) without GFR inhibitors.

In certain embodiments, a DIM-related indole is used in combination with farnesyl transferase inhibitors, in particular, Tipifarnib (R11577) at an oral dose of 300-1800 mg once or twice a day, with proteosome inhibitors such as Bortezomib given intravenously from 0.25-2.0 mg/m², or with RAF inhibitor, Sorafenib (BAY 43-9006) at an oral dose of 50-500 mg twice a day.

IV. Therapy of HCMV-Associated Pre-Cancerous Conditions

HCMV-associated pre-cancerous, cell-proliferative disorders include prostatic intraepithelial neoplasia (PIN), and benign, non-familial colonic polyposis. These conditions, diagnosed by surgical biopsy, are generally treated with a combination of orally administered DIM-related indole and GFR inhibitor administered for 3-6 months. In a less preferred embodiment, a DIM related indole is used alone. Daily dosage is chosen at the highest well tolerated level for each agent.

Men with PIN, proven histologically on prostate biopsy, are treated for 3-6 months utilizing a MTD dose of DIM-related indole and a ATD of GFR inhibitor taken orally. Since individuals with these disorders are typically asymptomatic doses of DIM-related indole and GFR inhibitor are chosen to be the highest, well tolerated combined use doses. This is accomplished giving a daily oral dose of 200-500 mg/day of DIM-related indole in suitable formulation along with a daily oral dose of 25-1000 mg of Gefitinib. Alternatively, PTK787 [ZK222584] at 750 mg/day can be used in place of the Gefitinib. In one embodiment this involves formulated DIM administered at 250 mg twice daily and Gefitinib 250 mg once daily for 3-6 months. Optimally, in cases of PIN the serum PSA level will be followed every 3-6 months to indicate reduced disease activity. Appropriate doses for colonic polyposis would be formulated DIM 150-200 mg/day and Gefitinib at 25-350 mg/day mg/day for 3-6 months. In colonic polyposis, the Gefitinib is started at the lowest possible dose, demonstrated to not cause diarrhea and DIM-related indole is started subsequently at 200 mg/day for a period of 4 months.

In certain embodiments, a DIM-related indole is used in combination with farnesyl transferase inhibitors, in particular, Tipifarnib (R11577) at an oral dose of 300-1800 mg once or twice a day, with proteosome inhibitors such as Bortezomib given intravenously from 0.25-2.0 mg/m², or with aRAF inhibitor, e.g., Sorafenib (BAY 43-9006) at an oral dose of 50-500 mg twice a day.

V. Therapy of HCMV-Associated Cancerous Conditions

HCMV-associated cancerous conditions include prostate cancer, colon cancer, malignant glioma, Epstein-Barr virus negative Hodgkin's disease, and non-melanoma skin cancer. Combined oral use of DIM-related indoles and GRF inhibitors blocking HER, PDGF, and/or VEGF receptors can be given at the maximal tolerated dose for variable treatment periods, preferably, of at least 1 month. In men who experience rising serum PSA levels following prostatectomy, combined DIM-related indoles and GFR inhibitors can be administered with or without additional anti-androgen drugs such as bicalutamide (Casodex, Astra-Zeneca, UK). In cases of metastatic colon cancer or malignant glioma combined DIM-related indoles and GFR inhibitors are administered concomitantly with radiation therapy.

Treatment of HCMV-related cancer according to the present invention will include the spectrum of asymptomatic men with recurrence of prostate cancer following surgery indicated by a rising PSA to symptomatic men with known metastatic prostate cancer with painful metastasis. The combined use of DIM-related indole and GFR inhibitor is undertaken at moderate to high doses. Treatment of the serious long term threat to life supports the use of the highest tolerated combined doses. Typically a daily oral dose of 250-750 mg/day of DIM-related indole in suitable formulation is taken along with a daily oral dose of 25-1000 mg of Gefitinib or alternatively 750-1200 mg of PTK787 [ZK222584] (Novartis/Schering). Men with post-surgical prostate cancer and rising PSA's are treated for 3-12 months utilizing 300-750 mg/day of DIM-related indole in suitable formulation taken along with a daily oral dose of 25-1000 mg of Gefitinib or PTK787 [ZK222584] at 750-1000 mg or at well tolerated doses. Once adequate reduction of PSA has been achieved, DIM-related indole at a dose of 100-200 mg/day given with or without Resveratrol 25-1000 mg/day subsequently. For men with metastatic prostate cancer the maximal tolerated dose is given for 3-6 months. Following this subsequent use is determined based on PSA levels, clinical response, and radiologic evidence of tumor size. Typically, treatment includes formulated DIM administered at 150-750 mg twice daily and Gefitinib 25-750 mg once daily or alternatively, PTK787 [ZK222584] at 1200-1500 mg per day. The treatment approach to malignant glioma is similar to that for metastatic prostate cancer. Treatment includes intercurrent use of additional chemotherapy, formulated DIM administered at 150-750 mg twice daily, and Gefitinib 25-750 mg once daily or PTK787 [ZK222584] at 1200-1500 mg per day.

In certain embodiments, a DIM-related indole is used in combination with farnesyl transferase inhibitors, in particular, Tipifarnib (R11577) at an oral dose of 300-1800 mg once or twice a day, with proteosome inhibitors such as Bortezomib given intravenously from 0.25-2.0 mg/m², or with aRAF inhibitor, e.g., Sorafenib (BAY 43-9006) at an oral dose of 50-500 mg twice a day.

A summary of the dose ranges appropriate for combined uses of DIM-related indoles with GFR inhibitors is presented in Table 3.

TABLE 3

| | Treatment | Treatment | Treatment | Treatment | Treatment |
|---|---|---|---|---|---|
| | Category I | Category II | Category III | Category IV | Category V |
| Agent | Chemo- | Active | Benign cell | Pre-Cancer | Established |
| | prevention | Infection | proliferative | Disoders | Cancer |
| | | | disorders | | |
| DIM-related | MED | ATD | ATD | MTD | MTD |
| indole | | | | | |
| GFR inhibitor | MED/ATD | MED/ATD | ATD | ATD | MTD |
| Gene Therapy | (−) | (+/−)* | (+/−) | (+/−) | (+/−) |
| Antisense | | | | | |
| Chemotherapy | (−) | (+/−)* | (−) | (+/−) | (+/−) |
| Radiation | (−) | (−) | (−) | (+/−) | (+/−) |
| Therapy | | | | | |

*Use specified in De Clercq, 2004, J Clin Virol. 30: 115-33.

TABLE 4
Dose Ranges for Combined uses of DIM-Related Indoles and GFR Inhibitors

| | | Minimal | | Average | | Maximal | |
|---|---|---|---|---|---|---|---|
| | | Effective Dose | | Tolerated Dose | | Tolerated Dose | |
| | | Range | | Range (ATD) | | Range (MTD) | |
| Drug | Manufacturer | (MED) mg/day | | mg/day | | mg/day | |
| Formulated | BioResponse | 25-150 | | 150-500 | | 500-1000 | |
| DIM | | | | | | | |
| (BR-DIM) | | | | | | | |
| ZD1839 | AstraZeneca | 25-150 | | 150-350 | | 350-750 | |
| Gefitinib | | | | | | | |
| (Iressa) | | | | | | | |
| Lapatinib | GlaxoSmithKline | 175-500 | | 500-900 | | 900-1,800 | |
| GW- | | | | | | | |
| 572016 | | | | | | | |
| OSI-774 | OSI/DNA/ | 50-150 | | 150-200 | | 200-400 | |
| Erlotinib | Roche | | | | | | |
| (Tarceva) | | | | | | | |
| Imatinib | Novartis | 100-300 | | 300-400 | | 400-800 | |
| Myesylate | | | | | | | |
| (Gleevec) | | | | | | | |
| STI-571 | | | | | | | |
| CI-1033 | Pfizer | 10-100 | | 100-500 | | 500-700 | |
| Efalizumab | | 5-25 | | 25-75 | | 75-200 | |
| Xanelin | | | | | | | |
| EKB-569 | | | | | | | |
| PKI-166 | Novartis | 10-50 | | 50-100 | | 100-900 | |
| Semaxanib | Sugen | 10-50 | mg/m² | 50-100 | mg/m² | 100-200 | mg/m² |
| SU5416 | Pharma/ | | | | | | |
| | Pfizer | | | | | | |
| CEP-7055 | Sanofi- | 25-100 | | 100-400 | | 400-1000 | |
| | Synthelab | | | | | | |
| Pegaptanib | Pfizer/ | 0.3 (intra-vitreal | | 1 (intra-vitreal | | 3 (intra-vitreal | |
| (Maucagen) | Eyetech | injection q 6 wks) | | injection q 6 wks) | | injection q 6 wks) | |
| Vatalanib | Novartis/ | 750-1,000 | | 1,000-1250 | | 1250-1500 | |
| PTK787 | Schering | | | | | | |
| (ZK222584) | | | | | | | |

4.5 Methods of Combined Use of DIM Related Indoles, GFR Inhibitors, and Radiation Therapy for Improved Treatment of HCMV-Related Cancer.

The present invention also provides methods of treating HCMV-related cancers using a combination of a DIM-related indole and a GFR inhibitor. There have been no reports of successful human use of GFR inhibitor drugs or antibodies in conjunction with radiation therapy as radiation sensitizing agents in human cancer.

The combined use of DIM-related indoles and GFR inhibitors provides a new approach to enhancing tumor cell radiosensitivity. Radiation therapy provides the advantage of a localized therapy delivered precisely to the tumor. The use of orally active DIM-related indoles and GFR inhibitors of the present invention provides a convenient means for optimizing tissue levels off these agents coordinated with the timing of radiation treatments without the need for intravenous therapy. This treatment optimizes the immediate cytotoxic effect of radiation since cellular repair pathways are inhibited. Pre- and post-radiation therapy use of DIM-related indole and oral GFR inhibitor drug, is continued for 5-7 days after completion of radiotherapy to inhibit survival and regrowth of radiation resistant tumor cells. Since resistance to radiation induced cell death is mediated by the GFR signaling pathways, particulary the PKB/Akt cell survival pathway activated by growth factors, including EGF, PDGF, and VEGF, inhibition of these pathways by DIM-related indoles and GFR inhibitors is expected to provide for a more complete therapeutic response to radiation therapy.

Using locally metastatic prostate cancer as an example, DIM-related indoles and GFR inhibitors are utilized with or without the addition of additional chemotherapy in conjunction with tumor site directed radiation therapy. Typically, the combined use of DIM-related indoles and GFR's with radiation therapy allows a reduction in the total radiation dose and fewer radiation associated side effects including skin changes. Reductions of at least 30% from the typical maximal radiation dose of 7000 gy for prostate cancers are possible with this combined therapy. Reduced fractionation of the total radiation dose with fewer treatment sessions is also made possible.

In certain embodiments, an oral DIM-related indole, alone or in combination with oral GFR inhibitors, can be combined with other oral or intravenous chemotherapeutic agents. This includes use with farnesyl transferase inhibitors, in particular, Tipifarnib (R11577) at an oral dose of 300-1800 mg once or twice a day, with proteosome inhibitors such as Bortezomib given intravenously from 0.25-2.0 mg/m², or with RAF inhibitor, Sorafenib (BAY 43-9006) at an oral dose of 50-500 mg twice a day. In combined therapy with DIM-related indoles at their MTD (250-1000 mg/day), Gefitinib, or other suitable GFR inhibitor, is co-administered orally in a dose of 250-750 mg/day 2-3 hours prior to a radiation therapy treatment. Both oral Gefitinib and DIM-related indole are continued on a daily basis during a typical week long series of radiation therapy treatments. Alternatively, an additional orally active GFR inhibitor such as Imatinib Myesylate (Gleevek, Novartis Pharmaceuticals) at 100-600 mg/dose or PTK787 [ZK222584] (Novartis/Schering) at 750-1200 mg/dose is added once daily 2 hours prior to radiation therapy. Intravenous, monoclonal antibodies against GFR receptors may additionally be administered 2 hrs before radiation therapy. Typically, Trastuzumab (Herceptin, Genentech, Calif.) is administered intravenously at a dose of 2-4 mg/kg administered in a 30-90 intravenous infusion prior to radiation treatments. Ideally, based on computerized tomography and magnetic resonance imaging, “Gammaknife” or “Cyberknife” (Accuray, Inc., Sunnyvale, Calif.) radiation therapy technology is also used to concentrate and focus the radiation beam limiting the radiation exposure of normal tissue adjacent and distant to the tumor mass. In the interim “rest period” of one or more weeks before a subsequent series of radiation therapy, oral DIM-related indoles and GFR inhibitors are continued at a lowered daily dose. Typically, the DIM-related indole is taken twice a day at the ATD (150-500 mg/dose) and Gefitinib is taken once a day at the ATD (150-350) mg/day.

Radiation therapy is site-directed with local and systemic side effects related to the total delivered dose measured in Gray (Gy) units, defined as one joule per kilogram of delivered energy. Clinically, one-hundredth Gy, or one cGy, is used as the unit of dose delivered. Total cumulative doses of 2,000 to 6,000 cGys result in severe complications after 5 years depending on the anatomic site irradiated. It is an object of the present invention to provide a means of reducing total radiation dose while seeking cure or long term control of metastatic cancer at a lower total cGy dose. Technology for optimizing radiation therapy dose is taught in U.S. Pat. No. 6,477,229, herby expressly incorporated by reference in its entirety. Addition of a DIM-related indole or combined DIM-related indole and GFR inhibitor therapy is expected to reduce the total required radiation dose for cancer cure or local control. The combined DIM-related indole/GFR inhibitor adjunctive therapy are thus radiosensitizers which increase the therapeutic response making tumor cell more susceptible to the effects of radiations, permiting lower radiation doses and reducing risks of radiation related side effects.

TABLE 5
Summary of combined uses of DIM-related indoles and GFR inhibitor
therapy for HCMV related diseases:

| | | Agent | | | | |
|---|---|---|---|---|---|---|
| | | DIM- | | Anti- | Mono- | |
| HCMV-related | | related | GFR | Viral | clonal | Radiation |
| Use or Condition | | Indole | Inhbitor | Agent | Antibody | Therapy |
| I | Prophylaxis | (+) | (+/−) | (−) | (−) | (−) |
| | Latent | | | | | |
| | Infection | | | | | |
| II | Active | (+) | (+/−) | (+) | (−) | (−) |
| | Infection | | | | | |
| III | Benign Cell | (+) | (+) | (+/−) | (−) | (−) |
| | Proliferative | | | | | |
| | Disorder | | | | | |
| IV | Precancerous | (+) | (+) | (+/−) | (+/−) | (−) |
| | Disorder | | | | | |
| V | Recurrent | (+) | (+) | (+/−) | (+/−) | (+/−) |
| | Cancer | | | | | |
| VI | Metastsatic | (+) | (+) | (+/−) | (+/−) | (+/−) |
| | Cancer | | | | | |

(+) = therapy utilized
(−) = therapy not utilized
(+/−) = therapy optionally utilized

4.6 Administration and Dosage

In preferred embodiments, certain combinations of DIM-related indoles, e.g., DIM, and a GFR inhibitor in topical delivery systems, parenteral delivery systems, oral delivery systems, and simultaneous delivery by multiple routes provides therapeutic efficacy more than the additive efficacy of each agent used alone at maximal dose. Therefore, methods involving combined use of a DIM-related indole and a GFR inhibitor at less than their maximal doses increase both the safety and efficacy of DIM-related indoles and GFR inhibitors in HCMV-related conditions.

Improved efficacy results in a shorter duration of required therapy than with individual agents used alone. Combined use allows a reduction in dose or concentration of each component in topical formulations. Combined use improves the long term therapeutic result with a lower rate of recurrence due to persisting virally infected cells. Combined use with lowered dose and duration of use minimizes toxicity. Combined use with higher dose improves the efficacy of dose-dependent therapy of HCMV-related cancer therapy to overcome cancer cell resistance to each individual agent therapy alone.

In methods involving the oral use of one or more DIM-related indoles, e.g., DIM, and a GFR inhibitor, the oral delivery of indole is facilitated and accomplished according to formulations and methods described in U.S. Pat. No. 6,086,915, incorporated by reference in its entirety.

When combined with HCMV antiviral drugs for the treatment of HCMV infections and for organ transplant vasculopathy, DIM-related indoles and GFR inhibitors can be added to established protocols. For example, DIM related indoles and GFR inhibitors can be used in conjunction with gancyclovir and anti-HCMV immunoglobulins in prophylaxis and treatment before and after organ transplantation (Bonaros et al., 2004, Transplantation 77:890-7).

The treatment of cutaneous, oral, and genital manifestations of HCMV infection with an oral DIM-related indole, e.g., DIM, is facilitated by topical, intravenous, intra-lesional, and aerosol application of DIM-related indoles in specific relative doses to the simultaneous administration of a GFR inhibitor. These therapies include production of tinctures, creams, vaginal or rectal suppositories, eye drops, emulsions for intravenous use, and injectable suspensions to deliver synergistic amounts of these agents. The present invention is based on an expected enhanced response in HCMV-related cancer cells when one or more DIM-related indoles, e.g., DIM, is used in combination with a GFR inhibitor.

4.7 Pharmaceutical Compositions

The pharmaceutical compositions according to the present invention preferably comprise one or more pharmaceutically acceptable carriers and the active constituents, i.e., a DIM-related indole and a GFR inhibitor. The carrier(s) must be “acceptable” in the sense of being compatible with the other ingredients of the composition and not deleterious to the recipient thereof.

It will be appreciated that the amounts of DIM or other DIM-related indole and/or a GFR inhibitor, required for said treatments will vary according to the route of administration, the severity of the HCMV-related disease, age, and file history of the subject, the galenic formulation of the pharmaceutical composition, etc.

Preferably, the DIM used in the invention has been processed to enhance bioavailability, as is described in United States Patent Application No. 6,086,915, incorporated herein by reference in its entirety; however any suitable preparation of pure diidolylmethane can be used in the methods and compositions of the invention.

In general, a suitable (therapeutically effective) amount of Diindolylmethane is preferably administered in an absorption enhancing formulation, as described in United States Patent Application No. 6,086,915, at 150-750 mg per day as a suspension of microparticles in a starch carrier matrix. Structurally-related, synthetically-derived, substituted diindolylmethane's, as described by Jong (U.S. Patent Application Publication No. 2004/0043965) are administered according to the present invention in an acceptable formulation for oral administration in a dose of 10-400 mg/day. Preferably, these substituted diindolylmethanes are administered in an absorption-enhanced formulation at a dose of 50 to 250 mg/day. The actually administered amounts of DIM or a substituted diindolylmethane may be decided by a supervising physician. The DIM-related indole of the invention is preferably administered in combination with an GFR antagonist administered by either oral, topical, or parenteral routes.

Typically, in the methods and compositions employing an GFR inhibitor, e.g., Iressa, Iressa would be employed in a dose of 50-500 mg/day, more preferably, 50-250 mg/day, or 50-100 mg/day. Alternatively, a low, effective dose of another GFR inhibitor such as OSI-774 (Erlotinib, Tarceva), CI 1033 [Parke-Davis Pharmaceutical Research (Ann Arbor, Mich.), PKI 166 [Novartis Pharma, AG (Basel, Switzerland)] or GW2016 would be employed at doses of 25-500 mg/day. As an example of such combined therapy, an absorption-enhanced formulation of DIM in a dose of 300 mg [75 mg actual DIM] is taken orally twice daily along with a dose of 100 mg of Iressa (ZD1839, Gefitinib) taken once daily. Further details of the clinical use of GFR inhibitors for combined use with DIM and/or DIM-related compounds are described in the following publications, incorporated herein by reference in its entirety (Janmaat et al., 2003, Oncologist 8:576-86; and Janmaat et al., 2003, Drugs Today (Barc) 39 Suppl C:61-80).

Therapeutic formulations include those suitable for parenteral (including intramuscular and intravenous), topical, oral, vaginal, rectal or intradermal administration, although oral administration for DIM is the preferred route. Thus, the pharmaceutical composition may be formulated as tablets, pills, syrups, capsules, suppositories, ophthalmic suspension, formulations for transdermal application, powders, especially lyophilized powders for reconstitution with a carrier for intravenous administration, etc.

The term “carrier” refers to a diluent, adjuvant, excipient, or vehicle with which the therapeutic is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; a glidant, such as colloidal silicon dioxide; a sweetening agent, such as sucrose or saccharin; and/or a flavoring agent, such as peppermint, methyl salicylate, or orange flavoring.

Therapeutic formulations suitable for oral administration, e.g., tablets and pills, may be obtained by compression or molding, optionally with one or more accessory ingredients. Compressed tablets may be prepared by mixing phytochemicals, and compressing this mixture in a suitable apparatus into tablets having a suitable size. Prior to the mixing, the DIM-related indole and/or GFR inhibitor may be mixed with a binder, a lubricant, an inert diluent and/or a disintegrating agent.

In a preferred embodiment, the DIM-related indole is mixed with a binder, such as microcrystalline cellulose, and a surfactant, such as sodium lauryl sulphate until a homogeneous mixture is obtained. Subsequently, another binder, such as polyvinylpyrrolidone (polyvidone), is transferred to the mixture under stirring with a small amount of added water. This mixture is passed through granulating sieves and dried by desiccation before compression into tablets in a standard tableting apparatus.

A tablet may be coated or uncoated. An uncoated tablet may be scored. A coated tablet may be coated with sugar, shellac, film or other enteric coating agents.

Therapeutic formulations suitable for parenteral administration include sterile solutions or suspensions of the active constituents. An aqueous or oily carrier may be used. Such pharmaceutical carriers can be sterile liquids, such as water and oils, including those of petroleum, animal, vegetable or synthetic origin, such as peanut oil, soybean oil, mineral oil, sesame oil and the like. Formulations for parenteral administration also include a lyophilized powder comprising phytochemical that is to be reconstituted by dissolving in a pharmaceutically acceptable carrier that dissolves said phytochemical. Parenteral administration also includes a stable emulsion of DIM designed for intravenous use. Ideally, the emulsion prevents the early removal of DIM from the circulation due to early uptake by the reticulo-endothelial system allowing maximal cellular concentration of DIM in HCMV-infected cells or tumor tissue.

When the pharmaceutical composition is a capsule, it may contain a liquid carrier, such as a fatty oil, e.g., cacao butter.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. The composition can be formulated as a suppository, with traditional binders and carriers such as triglycerides.

In yet another embodiment, the therapeutic compound can be delivered in a controlled release system. In one embodiment, a pump may be used (see Langer, supra; Sefton, CRC Crit. Ref. Biomed. Eng. 1987, 14:201; Buchwald et al., Surgery 1980, 88:507; Saudek et al., N. Engl. J. Med. 1989, 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 1983, 23:61; see also Levy et al., Science 1985, 228:190; During et al., Ann. Neurol. 1989, 25:351; Howard et al., J. Neurosurg. 1989, 71:105).

Other controlled release systems are discussed in the review by Langer (Science 249:1527-1533 (1990)).

In one embodiment of the pharmaceutical composition according to the invention, the DIM-related indole and GFR inhibitor are comprised as separate entities. The entities may be administered simultaneously or sequentially.

The invention also provides a pharmaceutical pack or kit comprising one or more containers filled with one or more of the ingredients of the pharmaceutical compositions of the invention. This includes the combination of capsules for oral use and creams or gels for simultaneous topical application. Optionally associated with such container(s) can be a notice in the form prescribed by a governmental agency regulating the manufacture, use or sale of pharmaceuticals or biological products, which notice reflects approval by the agency of manufacture, use or sale for human administration.

A number of references have been cited, the entire disclosures of which are incorporated herein by reference.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

### 5. EXAMPLES

5.1 Example: Manufacture of Processed DIM for Enhanced Oral Bioavailability

Preparation of processed Diindolylmethane was accomplished according to the steps outlined in United States Patent Application No. 6,086,915, herein incorporated by reference in its entirety. Briefly, this included mixture of about 10-40% by final weight of Diindolylmethane with about 10-40% by final weight of vitamin E polyethylene glycol 1000 succinate (Vitamin-E-TPGS, Eastman Chemical), 2-20% by final weight, phosphatidyl choline (Phospholipon 50G, Rhone Poulenc) and 15-30% by final weight hexanol. This mixture was made homogeneous by mixing. The homogeneous mixture of indoles and other oil soluble substituents listed above was added to a solution of modified starch in water (Capsul Starch from National Starch, Inc.). The starch component forms from 30-70% of the final dry weight of the product. The well dispersed final combined mixture was then subjected to spray drying. The resultant product was a fine powder containing either Diindolylmethane contained within the starch particles.

5.2 Example: Manufacture of Capsules Containing Diindolylmethane

Capsules containing 150-300 mg of processed Diindolylmethane, as produced according to the steps described in Section 5.1, were made by mixing the processed Diindolylmethane with microcrystaline cellulose and placing the mixed powder into opaque gelatin capsules.

Capsules containing the combination of 150 mg of processed Diindolylmethane and 30 mg of Resveratrol from 300 mg of Regrape X (Interpharma Praha, CZ), were made by mixing the processed Diindolylmethane, Regrape X, with microcrystaline cellulose or rice flour excipient and placing the mixed powder into opaque gelatin capsules.

5.3 Example: Manufacture of DIM with GFR Inhibitor for Vaginal or Rectal Administration

In a heated vessel, 90 grams cetostearyl alcohol (Alfol 16/18, Vista) mixed with 10 cc Grapefruit Oil (Aldrich Chemical) was heated to 100° C. to which 5 gms of microcrystalline DIM, 10 gms of Silybin (LKT Labs, St. Paul, Minn.), and 10 gms of deferiprone (Ferriprox, Apotex Labs, Canada) were added with constant mixing to form a hot slurry. Alternatively, 90 grams cetostearyl alcohol (Alfol 16/18, Vista) is heated to 100° C. to which 5 gms of microcrystalline DIM is mixed and to which is added 2-5 grams of Gefintinib (Astra Zeneca), alone or together with 10 grams of ceramide or synthetic cerimide derivatives, C2 ceramide. In a second vessel 400 gms of IV Novata (Semi-synthetic Glyceride Suppository Base, Ashland Chemicals) was warmed to 40° C. with constant mixing. The well mixed slurry from the first vessel was added with continued mixing to the second vessel. The homogenized molted suppository material was formed into suppositories of 2 gms each and cooled. Glyceryl monsterate 10-50 gms was added to the molten mixture as needed to increase the firmness of the final suppositories.

5.4 Example: Manufacture of DIM with a GRF Inhibitor in a Penetrating Oil for Topical Administration

In a heated vessel, 500 cc of Grapefruit Oil (a source of concentrated Limonene) (Aldrich Chemical) was heated to 50° C. to which 7.5 gms of microcrystalline DIM, 5 gms of Erlotinib (Tarceva, OSI Phamaceuticals) were added with constant mixing. The mixture was cooled and transferred to 10 cc brown glass bottles equipped with glass-rod applicator tops.

5.5 Example: Manufacture of a Drug-Eluting Vascular Stent Providing Intra-Vascular Sustained Release of a DIM-Related Indole and a GFR Inhibitor

DIM and a VEGFR inhibitor drug (SU11248 or SU10944 [Sugen Pharma, CA], or PTK787 [Novartis/Schering]) are combined in a production process to coat a vascular stent for intravascular deployment during angioplasty treating atherosclerotic arterial occlusion. Methods of manufacture of drug-eluting stents applicable for use with a DIM-related indole, alone or in combination with a GFR inhibitor, are provided in US Patent Application Publication No. 20040185168. Alternatively, a process for adding DIM-related indoles and GFR drugs to coatings for vascular stents utilizes the techniques described in U.S. Pat. No. 6,569,195 by Yang et al.

5.6 Example: Synergistic Anti-HCMV Activity of DIM and GFR's in a Cell Culture Model

Cell culture studies are performed to establish activity of DIM-related indoles alone and in combination with GFR's as inhibitors of HCMV replication in an in vitro model relevant to human infection.

Cell culture and viral infection. Human embryonic lung (HEL) fibroblasts are cultured in Eagle's minimal essential media (EMEM) (Gibco, BRL) supplemented with 10% fetal bovine serum (FBS) plus antibiotics.

Towne strain HCMV (passages 36 to 40) is propagated in HEL fibroblasts as previously described (Kowalik et al., 1993, Proc. Natl. Acad. Sci. USA 90:1107-1111).

For infection, cells are grown to confluence and then are serum starved for 48 h in minimal essential media (MEM) plus antibiotics. Cells are infected with HCMV that had been purified through a sucrose cushion to eliminate cytokines and growth factor contamination. Seeding of cells using a multiplicity of infection of 2-5 PFU per cell is used.

The following compounds and combinations of DIM-related indoles and GFR inhibitors are tested utilizing the following dose ranges:

Diindolylmethane (0.25-50 micromolar)

Gefitinib (0.10-25 micromolar)

Erlotinib (0.10-25 micromolar)

Vatalanib (0.10-25 micromolar)

SU10944 (0.10-25 micromolar)

Resveratrol (0.25-50 micromolar)

Diindolylmethane (0.25-50 micromolar) plus Gefitinib (0.10-25 micromolar)

Diindolylmethane (0.25-50 micromolar) plus Erlotinib (0.10-25 micromolar)

Diindolylmethane (0.25-50 micromolar) plus Vatalanib (0.10-25 micromolar)

Diindolylmethane (0.25-50 micromolar) plus Resveratrol (0.25-50 micromolar)

At the indicated time after infection, cells are washed once and then are maintained in MEM plus antibiotics until they are harvested. Cells are pretreated for 1 h with the listed compound or combinations of compounds prior to infection. In addition, the compound or compounds are present during infection and subsequent incubation periods. Mock-infected samples were treated and harvested in the same manner as the infected samples except that MEM without virus is used during the infection. For all experiments, the time at which virus is first added to the cells is the zero hour.

Titer reduction assay. The levels of active virus during the culture period are determined using a Titer Reduction Assay. Titer reduction assays are performed as previously described Johnson et al., 1999, Antivir. Res. 41: 101-11). Briefly, confluent HEL fibroblasts are infected as described above in the presence of the inhibitor compound or combination of compounds. To maintain a stable concentration of inhibitors compounds, fresh media containing appropriate concentrations of compounds are added every 48 h. At day 6 postinfection, the supernatant is harvested and is used to perform an HCMV standard plaque assay in a 24-well plate using 1% methyl cellulose overlayer containing 1×MEM and 4% fetal bovine serum. HCMV plaque numbers are scored under an inverted microscope.

A significant decrease in viral titers is expected with Diindolylmethane and GFR inhibitior compounds when tested individually. A number of combined treatment conditions are exected to show additive and greater than additive (synergistic) decreases in viral titers. Results will demonstrate anti-HCMV activity of Diindolylmethane and GFR inhibitors individually which is amplified using simple combinations of the compounds.

5.7 Example: Synergistic Apoptosis Promoting Activity of DIM and GFR's in a Prostate Cancer Cell Culture Model

The objective of these studies is to determine the chemosensitivity of human prostate adenocarcinoma, LNCap cells to combinations of DIM-related indoles and GFR inhibitors. The hypothesis tested is that combined treatment with the two classes of compounds will be additive and synergistic in promoting growth arrest and apoptosis in vitro. The LNCap prostate cancer cell line is a well studied model of androgen-sensitive prostate cancer which is relevant to HCMV-related malignancy. Only an HCMV vector, and not other viruses, is able to transfect the LNCap cell line (Ruokonen et al., 1996, Biochem Biophys Res Commun. 218:794-6).

Cell Culture Methods

LNCap cells are obtained from the American Type culture Collection, Manassas, Va. To assess the chemosensitivity of LNCap cancer cells to single and combination treatment with DIM and GFR inhibitors, cells are sub-cultured under 5% CO₂ at 37° C. for 48 hrs to reach 80-90% confluence. All cells are grown and maintained as monolayers in 0.25 m tissue culture flasks (Sigma Scientific, St. louis, MO, USA) in RPMI 1640 containing 15 mM HEPES, and supplemented with 0.45% glucose (w/w), 5.0% FBS and 100 U·mL⁻¹ penicillin+100 mg·ml⁻¹ streptomycin. The cells are then harvested by gentle scraping with a cell scraper. The cell suspensions are then grown at a density of 2.5×10³ cells/well in 24-well microtiter plates (MTP) for 36 hr to allow adherence. The supernatants are discarded and the agents (DIM and/or GFR inhibitor) are added over a range of 5 concentrations in single and combination treatments. All treatments are in triplicates. All plates are then incubated at 37° C. in a humidified atmosphere of 5% CO₂ in air for a maximum of 72 hr. At 12, 24 and 36 hr of incubation, 100 μl of the supernatant from each well was gently aspirated and replenished with 100 μl of fresh media. The supernatants are stored at −37° C. until assayed for lactate dehydrogenase (LDH) enzyme activity. At 48 and 72 hr incubation, the cells are harvested by trypsinization with trypsin-EDTA, and processed for post-treatment metabolic activity using cell viability and apoptotic assays as described.

Assay Methods

Assays for evidence of DIM-related indole and GFR inhibitor induced growth arrest and cell death include the MTS assay and the Trypan Blue exclusion assay. Treatment-induced apoptosis is assessed using one of two independent assays, Annexin V-FITC assay and the DNA Fragmentation (TUNEL) assay. Conditions to be tested include the following utilizing the following dose ranges:

Diindolylmethane (0.25-50 micromolar)

Gefitinib (0.10-25 micromolar)

Erlotinib (0.10-25 micromolar)

Resveratrol (0.25-50 micromolar)

Diindolylmethane (0.25-50 micromolar) plus Gefitinib (0.10-25 micromolar)

Diindolylmethane (0.25-50 micromolar) plus Erlotinib (0.10-25 micromolar)

Diindolylmethane (0.25-50 micromolar) plus Resveratrol (0.25-50 micromolar)

The following briefly describes the assay to be employed.

MTS Assay:

MTS assay depends on mitochondrial enzyme reduction of MTS solution to detect and determine cell viability. The MTS cell proliferation assay is a calorimetric method for determining the number of viable cells in proliferation. It is composed of solutions of a tetrazolium compound [3-(4,5-dimethylthiazol-2-yl)-5-(3-carboxymethoxyphenyl)-2-(4-sulfophenyl)-2H-tetrazolium, inner salt; MTS] and an electron coupling reagent (phenazine ethosulfate; PES). MTS is bioreduced by the cells into formazan product that is soluble in cell culture medium. Following cell culture as described above, 100 μL of cells are harvested from each treatment group and added to a 96 MTP followed by addition of 20 μl of MTS (2.5 mg/mL: Sigma Chemical Co) stock solution to each well. After 2 hr incubation under standard conditions of 5% CO₂ and 37° C., the purple formazan product (indicative of reduction of MTS) is visible. The absorbance is read on Multiskan biochromatic automated microplate reader (Multiskan, DC) at 490 nm. The signal generated (color intensity) is directly proportional to the number of viable (metabolically active) cells in the wells. Relative cell numbers can therefore be determined based on the optical absorbance (optical density, OD) of the sample. The blank values are subtracted from each well of the treated cells and controls; and the mean and standard error for each treatment (singles and combination) are calculated relative to the control:

$$\% of viable cells = \frac{{A}_{T} - {A}_{B}}{{A}_{C} - {A}_{B}} ⨯ 100$$

where A_{C}=absorbance of the control (mean value): A_{T}=absorbance of the treated cells (mean value)

A_{B}=absorbance of the blank (mean value)

Trypan Blue Exclusion Assay

For the Trypan blue exclusion test, cells are treated and cultured as described. They are harvested and Trypan blue dye solution was added to the cell suspensions. Total cell counts and viable cell number (survival rate) are determined by a standard hemocytometer procedure. Live-viable cells are seen as colorless (impermeable to the dye due to intact cell membrane) and dead cells are seen as blue (permeable to dye due to disruption of cell membrane):

$$\% Live ( Recovery rate ) = \frac{Live cells counted}{Total cells counted} ⨯ 100$$

Annexin V-FITC Assay

Apoptosis-associated translocation of phosphatidylserine from the inner to the outer leaflet of the plasma membrane in GC27 and K833 cells was assessed with the use of FITC-labeled Annexin V, a calcium-dependent phospholipid-binding protein with a high affinity for phosphatidylserine; using AnnexinV-FITC Staining Kit (Boehringer Mannheim). Briefly, 100 μl aliquots of the previously prepared cell suspensions are centrifuged, and the cell pellets re-suspended in Annexin binding buffer, incubated with AnnexinV-FITC substrate; then cells are smeared onto microscope slides and either evaluated immediately with fluorescence microscope, or smears are fixed with 4% depolymerized paraformaldehyde and stored at −40° C. for later examination as previously described (Zhang et al, 1997, Bio Techniques 23:525-531). Percentage of apoptosis in the cells is quantified based on morphological and fluorescence characteristics of apoptotic cells as previously described (Cotter et al., Techniques in apoptosis; a user's guide. Portland Press Ltd London; 1994. p. 10-12.]. All tests are run in triplicates.

DNA Fragmentation (TUNEL) Assay

The presence of apoptosis was determined by terminal deoxynucleotidyl transferase (TdT)-mediated dUTP nick end labeling (TUNEL), using the ApopTag® kit (Boehringer Mannheim Co, Indianapolis, Ind.) as previously described [Kumi-Diaka et al., 1998, Biology of the Cell 90:349-354]. The kit reagents detect apoptotic cells in situ by specific end labeling and detection of DNA fragments produced by the apoptotic process. To perform the TUNEL assay, slides of the PBS suspended cells are fixed with 4% paraformaldehyde for 30 minutes. The cells (slides) are then permeabilized with Triton X-100 at 4° C. for 2 min; then flooded with TdT enzyme and digoxigenin-dUTP reaction buffer (TUNEL) reagent for 60 min in a humidity chamber at 37° C., washed with distilled water, incubated for 10 minutes with streptavidin-horseradish peroxidase complex. The stained mounted cells are examined at 100×, 200× and 400× magnification of the microscope (Olympus BH-2). Cell death is quantified by counting 150 cells in 5-7 separate fields of view per slide, and noting the percentage of apoptotic cells based on morphological appearance, as previously described (Cotter et al., Techniques in apoptosis; a user's guide. Portland Press Ltd London; 1994. p. 10-12).

Growth arrest, cell death, and apoptosis specific assays are expected to indicate additive and synergistic decreases in cell proliferation and promotion of apoptosis with combined treatment using DIM-related indoles and GFR inhibitors.

5.8 Use of Primary Cultures of Human Prostate Tumors to Demonstrate Synergistic Apoptosis Promotion In Vitro with the Combination of DIM and GFR Inhibitors

A protocol to establish the synergistic activity of DIM, Tarceva® (Erlotinib, [OSI Pharmaceuticals]), and/or Gefitinib (Iressa®, ZD1839 [Astra Zeneca]) based on the exposure of primary cultures of human tumors is designed. Iressa® and Tarceva® are orally active EGFR-TKI's (epidermal growth factor receptor tyrosine kinase inhibitors) which block signal transduction pathways which may contribute to chemotherapy and radiation resistant cancer. Other inhibitors of the epidermal growth factor receptor (EGFR) to be tested include CI 1033 [Parke-Davis Pharmaceutical Research (Ann Arbor, Mich.)], a quinazoline tyrosine kinase inhibitor different from Iressa, PTK787/ZK 222584 (PTK787) (Vatalanib, Schering/Novartis), an orally active anilino-phthalazine inhibitor of VEGFR types 1 and 2, SU 10944 (3-[5-methyl-2-(2-oxo-1,2-dihydro-indol-3-ylidenemethyl)-1H-pyrrol-3-yl]-proprionic acid, an orally active pyrrole indolinone inhibitor of VEGFR type 2, and PKI 166 [Novartis Pharma, AG (Basel)], a non-quinazoline EGFR antagonist. The effects of DIM alone and in combination on tumor growth are evaluated using the EVA/PCD (ex vivo apoptotic/programmed cell death) assay (Rational Therapeutics Cancer Evaluation Laboratories, Long Beach, Calif.) which has previously been shown to correlate with response, time to progression and survival in patients.

Serial dilutions of DIM alone and in combination EGFR inhibitor drugs are applied to biopsy specimens of colon and prostate cancers. The PIP kinase inhibitor wortmannin in combination with DIM and other agents is also used to assess the influence of agents on the Akt-related pathway of apoptosis. Dose-response curves are interpolated to provide 50% lethal concentrations (LC(50)). The degree of synergy (by median effect) and normalised Z-scores (raw scores converted to relative activity distributed around the mean) is then computed.

The primary assay for apoptosis-related loss of cell viability is the mitochondrial function assay [reduction of 3-(4,5-dimethylthiazol-2-yl)-5-(3-carboxymethoxyphenyl)-2-(4-sulfophenyl)-2H-tetrazolium (MTS)] using a MTS kit (Promega, Madison, Wis.). A minimum of 4 replicate wells per condition are studied and absorbance at 595 nm of the solution in individual wells is determined with a multi-well plate reader. Data is analyzed by plotting the mean and SD of cell viability versus concentration of DIM or GFR Inhibitors.

A secondary assay to confirm the apoptoic mechanism of cell death measuring nucleosomal leakage is utilized. This assay detects histones and DNA in cytoplasmic extract and utilizes Cell Death Detection ELISA Plus kit from Roche Molecular Biochemical (Mannheim, Germany). Results are determined by measuring absorbance at 405 nm with the multi-well plate reader.

Favorable interactions are anticipated for DIM combinations with EGF receptor antagonists. Tumor cultures will be analyzed for synergistic increases in apoptosis-related cell killing with combinations of DIM and EGF inhibitors, DIM and Zinc-binding histone deacetylase inhibitors (HDAC's), and with the combination of DIM, HDAC's, and EGF Inhibitors.

These primary human culture studies may support synergistic and possibly clinically beneficial interactions of DIM-related indoles and GFR inhibitors.

5.9 Example: In Vivo Demonstration of Synergistic Action of DIM and GFR Inhibitors in Transgenic Mice as Compared to Treatment with DIM Alone

Animal treatment models provide information that is often predictive of clinical efficacy in clinical trials (Bosland et al., 1999, Eur Urol. 35:459-63). The present study utilizes transplanted tumors from the TRAMP-C2 prostate cancer cell line, considered androgen independent, and therefore relevant to advanced human prostate cancer (Foster et al., 1997, Cancer Res. 57:3325-30). Prostate cancer (PC) is a HCMV associated cancer (Samanta et al., 2003, J Urol. 170:998-1002) which has been shown in humans to be preceded by a chronic state of inflammation accompanied by infiltration of precancerous tissue by monocytes and macrophages (Anim 1998, Acta Histochem. 100:439-49). In addition the infiltrating monocytes/macrophages in benign prostatic hypertrophy have been shown to be positive for HCMV DNA (Stapleton et al., 1996, J Urol. 156:542-5).

This study of PC in animals is undertaken to demonstrate that orally administered DIM and Gefitinib induce additive and synergistic inhibition of growth of transplanted TRAMP-C2 prostate cancer cells growing in C57BL/6 mice. The C57BL/6 mouse is an established in vivo model for pre-clinical testing of anti-cancer therapeutics (Voeks, 2002, Urol Oncol. 7:111-8). The study documents the potential therapeutic effects of oral Absorption-Enhanced DIM used alone and in combination with an orally active GFR inhibitor like Gefitinib in an in vivo model. METHODS: TRAMP-C2 cells, a mouse PC cell line are obtained from the American Type Culture Collection (Rockville, Md.). Six week old male C57BL/6 mice are purchased from Harlan Laboratories (Harlan, USA). TRAMP-C2 cells are suspended in media and aliquots are injected into the flank of male C57BL/6 mice using a 27 gauge needle. Two weeks after tumor transplantation when tumors appear, mice are divided in six groups treated as indicated:

1. Control Diet

2. Absorption enhanced DIM at low dose (10 mg/kg/day)

3. Absorption enhance DIM at low dose (10 mg/kg/day) plus Gefitinib at low dose (20 mg/kg/day)

4. Absorption enhanced DIM at high dose (100 mg/kg/day)

5. Absorption enhance DIM at high dose (100 mg/kg/day) plus Gefitinib at low dose (20 mg/kg/day)

6. Oral Gefitinib alone at low dose (20 mg/kg/day)

7. Oral Gefitinib alone at high dose (200 mg/kg/day)

Animals are dosed by gavage once daily, 5 days per week. Tumor volumes are measured weekly for 4 weeks following transplantation using calipers. Later, the tumors are removed, their final weights and volumes are measured. Tumor tissue, liver tissue, intestinal tissue, and kidney tissue are prepared for histological studies. Blood samples are obtained. Tumor volumes in mm² are determined during the study using the formula π/6×larger diameter×(smaller diameter)². The data are expressed as the increase or decrease in tumor volume in mm³. Histological studies are performed to assess the contribution of apotosis promotion in the reduction of tumor volume in treated groups. Histological study of non-tumor tissue is used to assess treatment-related toxicity.

RESULTS: DIM and Gefitinib used alone is expected to show inhibition of tumor growth as seen by diminished tumor volumes in treatment groups compared to the control group. Combined treatment with both DIM and Gefitinib is expected to demonstrate a greater inhibition of tumor growth than with treatment using either agent alone. Histological examination of tumors from treated groups will reveal increased apoptosis and decreased cell proliferation, compared with the control group. Body weights in the treatment groups will remain within 10% of the control group of mice. No toxic effects, comparable to those seen at the high dose of Gefitinib (200 mg/kg/day) when used alone, is expected to be seen at the low dose groups of Gefitinib (20 mg/kg/day) used in combination with DIM.

CONCLUSIONS: An additive and/or synergistic anti-tumor action of the combination of DIM and Gefitinib in this mouse model will indicate potential for the combined therapeutic use of DIM-related indoles and GFR inhibitors, including Gefitinib, in human malignancies.

5.10 Example: Combined Oral DIM and Oral Iressa® Treatment in a Patient with Metastatic Prostate Cancer to Overcome Cancer Cell Resistance to Radiation Therapy

A 60-year-old male patient suffering from a local recurrence in pelvic lymph nodes 1 yr following retropubic prostatectomy for adenocarcinoma of the prostate gland is used as a case study. In recurrent prostate cancer, total radiation dose delivered to the pelvis including the local recurrence and surrounding tissue is typically 5-6000 cGy. This is accompanied by a high risk of radiation induced protitis, cystitis, and other complications. Reduction in fractionated and total radiation dose is a goal of improved therapy. In the following example the use of oral DIM and oral Gefitinib is expected to permit a reduction in treatment radiation dose from the typical 500 cGy per treatment to 300 cGy per treatment. A reduced total radiation dose will help reduce short and long term radiation-related side effects.

On the first treatment day the patient, weighing 70 kg, undergoes Computerized Tomography (CT) with intravenous omnipaque to enhance the 3 dimensional definition of the recurrent tumor mass.

On the afternoon of the first treatment day, the patient undergoes initial radiation therapy preceded by an oral dose of Iressa® 500 mg (two 250 mg capsules) and oral DIM given at 6 mg/kg (four 75 mg DIM Capsules). Each oral agent is given with 8 oz of water on an empty stomach. 2 hours after the oral doses of DIM and Iressa®, a 300-cGy radiation treatment is delivered to the tumor site by the Cyberknife (Acurray Inc, Sunnyvale, Calif.). Following this oral DIM (four 75 mg DIM capsules) and Iressa® (250 mg per dose) are continued every 8 hours. The two-week treatment course for the patient is summarized in the following chart:

TABLE VII

| Treatment | | | | | | | | | |
|---|---|---|---|---|---|---|---|---|---|
| Component | Day 1 | Day 2 | Day 3 | Day 4 | Day 5 | Day 6 | Day 7 | . . . | Day 12 |
| CT with | X | | | | | | | | |
| I.V. Contrast | | | | | | | | | |
| Pre-Radiation | X | X | X | X | X | | | | |
| Oral Iressa | | | | | | | | | |
| 500 mg | | | | | | | | | |
| Pre-Radiation | X | X | X | X | X | | | | |
| Oral DIM | | | | | | | | | |
| 300 mg | | | | | | | | | |
| Cyberknife | X | X | X | X | X | | | | |
| Radiation Tx | | | | | | | | | |
| Post-Radiation | X | X | X | X | X | X | X | X | X |
| Oral DIM | | | | | | | | | |
| 150 mg q 8 hrs | | | | | | | | | |
| Post-Radiation | X | X | X | X | X | X | X | X | X |
| Oral Iressa | | | | | | | | | |
| 250 mg q 8 hrs | | | | | | | | | |

The reduced total radiation dose through use of DIM and Gefitinib is expected to reduce radiation cystitis and protitis. The specific activities of DIM and Iressa® are believed to interact to inhibit anti-apoptotic tumor cell mechanisms of radio-resistance to achieve more complete tumor cell death in metastatic adenocarinoma of the prostate gland.

5.11 Clinical Study of Absorbable DIM Alone or Absorbable DIM in Combination with Gefitinib in Men with Prostatatic Intraepithelial Neoplasia

Introduction: Prostatic intraepithelial neoplasia (PIN) is the histologic lesion most strongly associated with prostate cancer and has been established to be a pre-malignant lesion. The prevalence of PIN and high grade prostatic intra-epithelial neoplasia (HGPIN) based on biopsy studies varies from 11-25% of biopsied men, depending on the selection criteria for biopsy (Feneley et al., 1997, Prostate Cancer Prostatic Dis. 1:79-83). Since no effective medical therapy is known, men who receive the diagnosis of PIN or HGPIN following prostate biopsy are offered no treatment. They are followed with serial PSA levels and given repeat prostate biopsies. Increased expression and activity of GFR's has been documented in PIN as well as prostate cancer (Lorenzo et al., 2003, Clin Prostate Cancer 2:50-7). DIM has been shown to inhibit growth of transplanted human prostate cancer in animals (Nachshon-Kedmi M et al., 2004, Prostate 61:153-60). Therefore, based on the need for safe and effective early intervention to prevent progression of PIN and HGPIN to prostate cancer, a Phase II study of GFR inhibitor (Gefitinib, Iressa), DIM, and the combination of GFR inhibitor and DIM is undertaken. Alternatively, the study protocol can be amended to substitute PTK787/ZK 222584 (PTK787) (Vatalanib, Schering/Novartis) at a dose of 750-1200 mg/day, divided into twice daily doses, for Gefitinib.

Study Objectives:

This study will undertake treatment of men with elevated PSA and a prostate needle biopsy which shows PIN or HGPIN. The objective and primary endpoints of the study will be to demonstrate a treatment response based on serial PSA determinations, and repeat prostate biopsy. The secondary endpoints of the study will be to document tissue levels of DIM and Gefitinib (or Vatalanib) in prostate tissue, changes in levels of phospho Akt immunohistochemistry on repeat biopsy tissue, and determination of the safety of long term combined DIM and Gefitinib treatment.

Study Plan:

A Phase II, randomized, double-blind, placebo contolled trial of DIM, Gefitinib (or Vatalanib), and DIM plus Gefitinib (or Vatalanib) in men with elevated PSA and prostate biopsy positive for PIN or HGPIN is initiated. The study will enroll 140 subjects to be randomized to one of 4 groups:

1. Placebo DIM twice a day. Placebo Iressa (or Vatalanib) once a day.

2. Oral DIM 200 mg twice a day. Placebo Iressa (or Vatalanib) once a day

3. Oral Gefitinib (Iressa) 250 mg once a day (or Vatalanib 750 mg twice daily). Placebo DIM twice a day.

4. Oral DIM 200 mg twice a day and oral Gefitinib (Iressa) 250 mg once a day (or Vatalanib 750 mg twice daily).

The study is designed to require 100 evaluable subjects. An evaluable subject will satisfy entrance requirements (elevated PSA and histologic diagnosis of PIN or HGPIN on ultrasound-guided biopsy) and exit requirements (serial PSA levels at 2, 4 and 6 months, and repeat ultrasound-guided prostate biopsy of positive prostate quadrant at 6 months).

Excess enrollment is planned to allow for subject discontinuation and possible treatment related side-effects.

Tissue obtained for repeat biopsy will include both paraffin-embedded and frozen cores. Frozen cores will be used to assess DIM and Gefitinib levels in prostate tissue. Paraffin-embedded tissue will be used for phospho Akt immunohistochemistry (Cell Signalling Systems, MA).

Laboratory Correlates:

PSA velocity. Free and total PSA will be measured at baseline and at 2 month intervals after randomization. Repeat PSA measurement will occur prior to the end of study biopsy.

Statistical Considerations:

Serum PSA (free and total) will be measured as a continuous variable. PSA velocity (change from baseline) will be compared across dose groups using a linear mixed effects model. Inferential testing will focus on whether or not PSA velocity differs significantly by treatment group.

Histology of primary biopsy and repeat prostate biopsy will be compared. Tissue diagnosis of HGPIN, PIN, and Prostate will be treated as dichotomous endpoints (0=absent; 1=present) that will be compared between groups using Fisher's exact test.

Results: Anticipated results will include treatment related reductions in PSA levels in the treatment versus placebo groups. Combined DIM and Gefitinib (or Vatalanib) treatment is expected to show additive or synergistically additive reduction of PSA levels that will be greater than a 50% reduction from baseline PSA levels. Histologic changes in response to treatment will show normalization of the repeat prostate biopsy. The combined DIM and Gefitinib (or Vatalanib) treatment group is expected to show the most consistent and complete normalization of prostate histology throughout the combined treatment group. Reduction in tissue phopho-Akt histochemistry staining will be seen in all treatment groups with a more marked reduction in the combined DIM and Gefitinib (or Vatalanib) treatment group.

What is claimed is:

1. A method of preventing or treating a HCMV-related condition comprising administering to a subject in need thereof a therapeutically effective amount of one or more DIM-related indoles and one or more GFR inhibitors.

2. The method of claim 1, wherein said HCMV-related condition is selected from the group consisting of a HCMV active infection, a HCMV-associated benign proliferative disorder, a HCMV-associated pre-cancerous condition and a HCMV-associated cancerous condition.

3. The method of claim 2, wherein said HCMV active infection is selected from the group consisting of intra-uterine infection, HCMV associated diabetes, HCMV associated CNS infection, HCMV associated polyneuritis, HCMV associated myelitis, and heterophile-negative mononucleosis syndrome, HCMV associated carditis, HCMV associated hepatitis, HCMV associated inflammatory bowel disease.

4. The method of claim 2, wherein said HCMV-associated benign proliferative disorder is selected from the group consisting of post-transplant intimal hyperplasia, HCMV-related atherosclerosis, HCMV associated post-allograft organ transplant vasculopathy, HCMV-associated macular degeneration, inflammatory bowel disease, arterial restenosis following angioplasty, vascular graft associated intimal hyperplasia in renal failure, idiopathic pulmonary fibrosis, and chronic interstitial nephritis.

5. The method of claim 2, wherein said HCMV-associated pre-cancerous condition is selected from the group consisting of prostatic intraepithelial neoplasia, cervical intraepithelial neoplasia, and benign, non-familial colonic polyposis.

6. The method of claim 2, wherein said HCMV-associated cancerous condition is selected from the group consisting of prostate cancer, cervical cancer, colon cancer, and non-melanoma skin cancer.

7. The method of claim 1, wherein the one or more DIM-related indoles are selected from the group consisting of:
a compound of formula I: 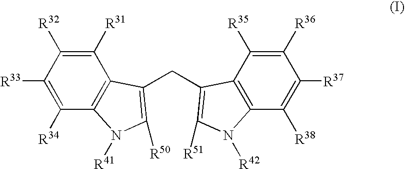
wherein R³² and R³⁶ are substituents independently selected from the group consisting of hydrogen, hydroxyl, and methoxy, and ethoxycarbonyl groups,
R³³ and R³⁷ are substituents independently selected from the group consisting of hydrogen, hydroxyl, and methoxy,
R³¹, R³⁴, R³⁵, R³⁸, R⁴¹, and R⁴² are hydrogen, and
R⁵⁰, R⁵¹ are either hydrogen or methyl;
a compound of formula II: 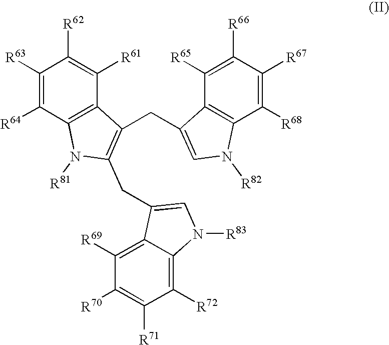
wherein R⁶², R⁶³, R⁶⁶, R⁶⁷, R⁷⁰, and R⁷¹ are substituents independently selected from the group consisting of hydrogen, hydroxyl, and methoxy, and
R⁶¹, R⁶⁴, R⁶⁵, R⁶⁸, R⁶⁹, R⁷², R⁸¹, R⁸², and R⁸³ are hydrogen;
a compound of formula (III): 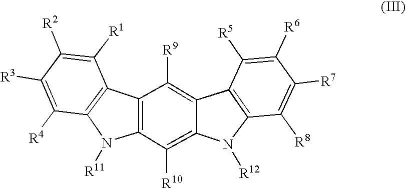
wherein R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, and
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
with the provisos that at least one of R¹, R², R³, R⁴, R⁵, R⁶, R⁷, R⁸, R⁹, R¹⁰, R¹¹ and R¹² is other than hydrogen, and when R¹, R², R³, R⁴, R⁵, R⁶, R⁷, and R⁸ are selected from hydrogen, halo, alkyl and alkoxy, then R¹¹ and R¹² are other than hydrogen and alkyl;
a compound of formula (IV): 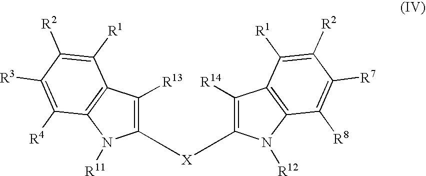
wherein R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
R¹³ and R¹⁴ are definied as for R¹, R², R³, R⁴, R⁵, R⁶, R⁷, and R⁸, with the proviso that at least one of R¹³ and R¹⁴ is other than hydrogen, and
X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹²; and
a compound of formula (V): 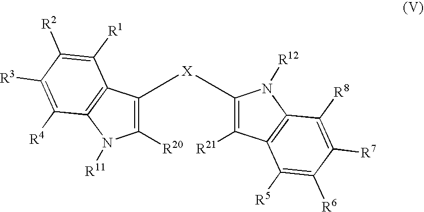
wherein R¹, R², R³, R⁴, R⁵, R⁶, R⁷, R⁸, R¹¹, R¹², and X are defined as for compounds of formula (III), and
R²⁰ and R²¹ are defined as for R¹, R², R³, R⁴, R⁵, R⁶, R⁷, and R⁸.

8. The method of claim 1, wherein the one or more DIM-related indoles are selected from the group consisting of diindolylmethane, hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

9. The method of claim 1, wherein the one or more GFR inhibitors is a GFR-specific small molecule drug or an GFR specific antibody.

10. The method of claim 9, wherein the small molecule drug is selected from the group consisting of gefitinib, ZD6474, erlotinib, lapatinib, GW-2016, imatinib myesylate, EKB-569, cancertinib, semaxanib, SU11248, SU6669, vatalanib, PKI-166, and CEP-7055.

11. The method of claim 9, wherein the small molecule drug is selected from the group consisting of SU10944 and pegaptanib.

12. The method of claim 9, wherein the GFR specific antibody is selected from the group consisting of cetuximab, trastuzumab, MDX-210, ABX-EGF, TheraCIM, panitumumab, EMD-72000, bevacizumab, and ranibizumab.

13. The method of claim 1, wherein said therapeutically effective amount of one or more DIM-related indoles and one or more GFR inhibitors is administered with a differentiation promoting agent.

14. The method of claim 13, wherein said differentiation promoting agent is selected from the group consisting of vitamin D, calcitriol, vitamin A, a retinoid derivative, and a macrophage colony stimulating factor.

15. The method of claim 1, wherein said therapeutically effective amount of one or more DIM-related indoles and one or more GFR inhibitors is administered with one or more of a farnesyl transferase inhibitor, a proteosome inhibitor, or a RAF inhibitor.

16. The method of claim 1, wherein the one or more DIM-related indoles and one or more GFR inhibitors are administered simultaneously.

17. The method of claim 1, wherein the one or more DIM-related indoles and one or more GFR inhibitors are administered within a short time of one another.

18. The method of claim 1, wherein the one or more DIM-related indoles are administered orally.

19. The method of claim 1, wherein the one or more DIM-related indoles and one or more GFR inhibitors are administered with a HCMV anti-viral drug selected from the group consisting of ganciclovir, valganciclovir, cidofovir, and phosphocarnet.

20. The method of claim 1, wherein the one or more DIM-related indoles and one or more GFR inhibitors are administered with resveratrol.

21. The method of claim 1, further comprising administering a radiation therapy regimen sufficient to treat a HCMV-associated condition.

22. The method of claim 21, wherein said radiation therapy comprises topical irradiation with ultraviolet radiation or x-rays.

23. The method of claim 1, further comprising administering a photodynamic therapy regimen sufficient to treat a HCMV-associated condition.

24. A method of preventing or treating a HCMV infection comprising administering to a subject in need thereof a therapeutically effective amount of one or more DIM-related indoles.

25. The method of claim 24, wherein the HCMV infection is a latent infection, active infection, or an HCMV-related benign proliferative state.

26. The method of claim 24, wherein said HCMV-associated benign proliferative disorder is selected from the group consisting of post-transplant intimal hyperplasia, HCMV-related atherosclerosis, HCMV associated post-allograft organ transplant vasculopathy, neovascular age-related macular degeneration, inflammatory bowel disease, arterial restenosis following angioplasty, vascular graft associated intimal hyperplasia in renal failure, idiopathic pulmonary fibrosis, and chronic interstitial nephritis.

27. The method of claim 24, wherein the one or more DIM-related indoles are selected from the group consisting of:
a compound of formula I: 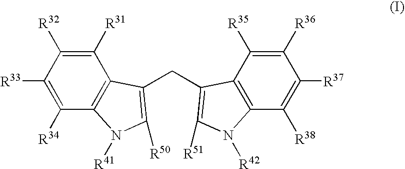
wherein R³² and R³⁶ are substituents independently selected from the group consisting of hydrogen, hydroxyl, and methoxy, and ethoxycarbonyl groups,
R³³ and R³⁷ are substituents independently selected from the group consisting of hydrogen, hydroxyl, and methoxy,
R³¹, R³⁴, R³⁵, R³⁸, R⁴¹, and R⁴² are hydrogen, and
R⁵⁰, R⁵¹ are either hydrogen or methyl;
a compound of formula II: 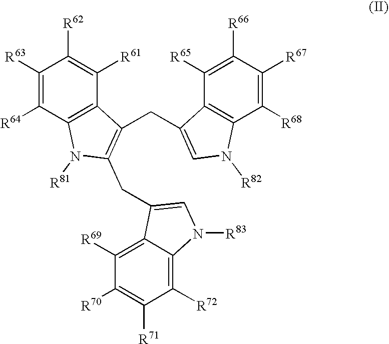
wherein R⁶², R⁶³, R⁶⁶, R⁶⁷, R⁷⁰, and R⁷¹ are substituents independently selected from the group consisting of hydrogen, hydroxyl, and methoxy, and
R⁶¹, R⁶⁴, R⁶⁵, R⁶⁸, R⁶⁹, R⁷², R⁸¹, R⁸², and R⁸³ are hydrogen;
a compound of formula (III): 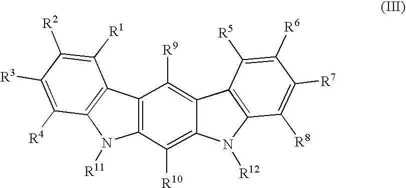
wherein R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, and
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
with the provisos that at least one of R¹, R², R³, R⁴, R⁵, R⁶, R⁷, R⁸, R⁹, R¹⁰, R¹¹ and R¹² is other than hydrogen, and when R¹, R², R³, R⁴, R⁵, R⁶, R⁷, and R⁸ are selected from hydrogen, halo, alkyl and alkoxy, then R¹¹ and R¹² are other than hydrogen and alkyl;
a compound of formula (IV): 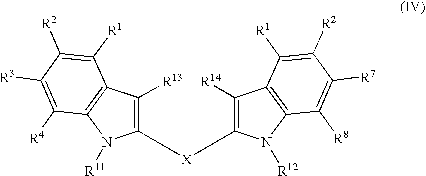
wherein R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
R¹³ and R¹⁴ are defined as for R¹, R², R³, R⁴, R⁵, R⁶, R⁷, and R⁸, with the proviso that at least one of R¹³ and R¹⁴ is other than hydrogen, and
X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹²; and
a compound of formula (V): 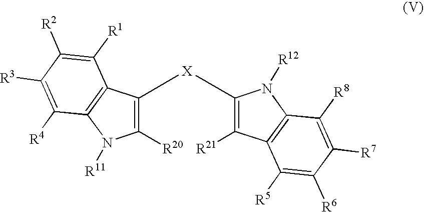
wherein R¹, R², R³, R⁴, R⁵, R⁶, R⁷, R⁸, R¹¹, R¹², and X are defined as for compounds of formula (III), and
R²⁰ and R²¹ are defined as for R¹, R², R³, R⁴, R⁵, R⁶, R⁷, and R⁸.

28. The method of claim 24 where the one or more DIM-related indoles are selected from the group consisting of diindolylmethane, hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

29. The method of claim 24, wherein the one or more DIM-related indoles are administered orally.

30. The method of claim 24, wherein said therapeutically effective amount of one or more DIM-related indoles is administered with a differentiation promoting agent.

31. The method of claim 30, wherein said differentiation promoting agent is selected from the group consisting of vitamin D, calcitriol, vitamin A, a retinoid derivative, and a macrophage colony stimulating factor.

32. The method of claim 24, wherein said therapeutically effective amount of one or more DIM-related indoles is administered with one or more of a farnesyl transferase inhibitor, a proteosome inhibitor, or a RAF inhibitor.

33. The method of claim 24, wherein said therapeutically effective amount of one or more DIM-related indoles is administered with a HCMV anti-viral drug selected from the group consisting of ganciclovir, valganciclovir, cidofovir, and phosphocamet.

34. The method of claim 24, wherein said therapeutically effective amount of one or more DIM-related indoles is administered with resveratrol.

35. A method of preventing or treating neovascular age-related macular degeneration comprising administering to a subject in need thereof a therapeutically effective amount of one or more DIM-related indoles.

36. The method of claim 35, wherein the neovascular age-related macular degeneration is not associated with an HCMV infection.

37. The method of claim 35, wherein the one or more DIM-related indoles are selected from the group consisting of:
a compound of formula I: 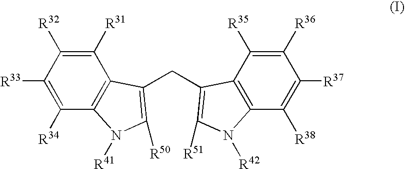
wherein R³² and R³⁶ are substituents independently selected from the group consisting of hydrogen, hydroxyl, and methoxy, and ethoxycarbonyl groups,
R³³ and R³⁷ are substituents independently selected from the group consisting of hydrogen, hydroxyl, and methoxy,
R³¹, R³⁴, R³⁵, R³⁸, R⁴¹, and R⁴² are hydrogen, and
R⁵⁰, R⁵¹ are either hydrogen or methyl;
a compound of formula II: 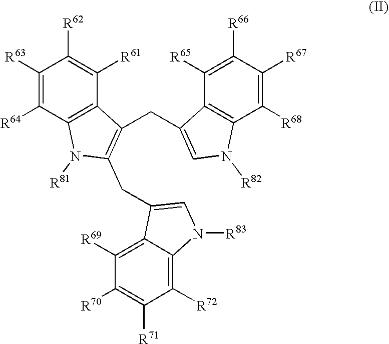
wherein R⁶², R⁶³, R⁶⁶, R⁶⁷, R⁷⁰, and R⁷¹ are substituents independently selected from the group consisting of hydrogen, hydroxyl, and methoxy, and
R⁶¹, R⁶⁴, R⁶⁵, R⁶⁸, R⁶⁹, R⁷², R⁸¹, R⁸², and R⁸³ are hydrogen;
a compound of formula (III): 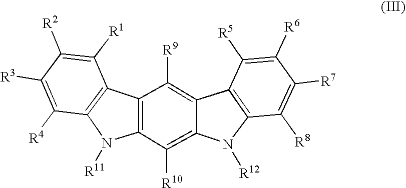
wherein R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, and
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
with the provisos that at least one of R¹, R², R³, R⁴, R⁵, R⁶, R⁷, R⁸, R⁹, R¹⁰, R¹¹ and R¹² is other than hydrogen, and when R¹, R², R³, R⁴, R⁵, R⁶, R⁷, and R⁸ are selected from hydrogen, halo, alkyl and alkoxy, then R¹ and R¹² are other than hydrogen and alkyl;
a compound of formula (IV): 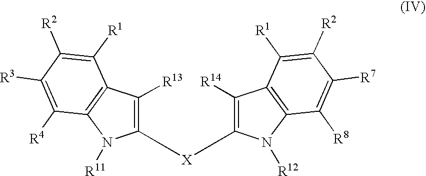
wherein R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
R¹³ and R¹⁴ are defined as for R¹, R², R³, R⁴, R⁵, R⁶, R⁷, and R⁸, with the proviso that at least one of R¹³ and R¹⁴ is other than hydrogen, and
X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹²; and
a compound of formula (V): 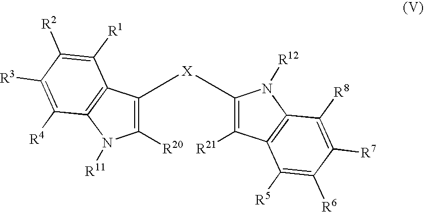
wherein R¹, R², R³, R⁴, R⁵, R⁶, R⁷, R⁸, R¹¹, R¹², and X are defined as for compounds of formula (III), and
R²⁰ and R²¹ are defined as for R¹, R², R³, R⁴, R⁵, R⁶, R⁷, and R⁸.

38. The method of claim 35 where the one or more DIM-related indoles are selected from the group consisting of diindolylmethane, hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

39. The method of claim 35, wherein said therapeutically effective amount of one or more DIM-related indoles is administered with one or more GFR inhibitors.

40. The method of claim 39, wherein the one or more GFR inhibitors is a GFR-specific small molecule drug or an GFR specific antibody.

41. The method of claim 40, wherein the small molecule drug is selected from the group consisting of gefitinib, ZD6474, erlotinib, lapatinib, GW-2016, imatinib myesylate, EKB-569, cancertinib, semaxanib, SU11248, SU6669, vatalanib, PKI-166, and CEP-7055.

42. The method of claim 40, wherein the small molecule drug is selected from the group consisting of SU10944 and pegaptanib.

43. The method of claim 40, wherein the GFR specific antibody is selected from the group consisting of cetuximab, trastuzumab, MDX-210, ABX-EGF, TheraCIM, panitumumab, EMD-72000, bevacizumab, and ranibizumab.

44. The method of claim 35, wherein the one or more DIM-related indoles are administered orally.

45. The method of claim 35 or 39, wherein said therapeutically effective amount of one or more DIM-related indoles is administered with a differentiation promoting agent.

46. The method of claim 45, wherein said differentiation promoting agent is selected from the group consisting of vitamin D, calcitriol, vitamin A, a retinoid derivative, and a macrophage colony stimulating factor.

47. The method of claim 35 or 39, wherein said therapeutically effective amount of one or more DIM-related indoles is administered with one or more of a farnesyl transferase inhibitor, a proteosome inhibitor, or a RAF inhibitor.

48. The method of claim 35 or 39, wherein said therapeutically effective amount of one or more DIM-related indoles is administered with a HCMV anti-viral drug selected from the group consisting of ganciclovir, valganciclovir, cidofovir, and phosphocarnet.

49. The method of claim 35 or 39, wherein said therapeutically effective amount of one or more DIM-related indoles is administered with resveratrol.

50. The method of claim 35 or 39, further comprising administering a photodynamic therapy regimen sufficient to treat neovascular age-related macular degeneration.

51. A pharmaceutical composition comprising a therapeutically effective amount of the combination of one or more DIM-related indoles and one or more GFR inhibitors.

52. The pharmaceutical composition of claim 51 where the one or more DIM-related indoles are selected from the group consisting of:
a compound of formula I: 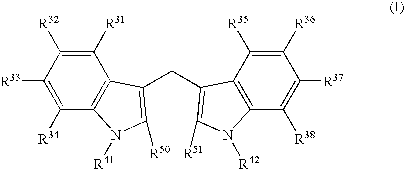
wherein R³² and R³⁶ are substituents independently selected from the group consisting of hydrogen, hydroxyl, and methoxy, and ethoxycarbonyl groups,
R³³ and R³⁷ are substituents independently selected from the group consisting of hydrogen, hydroxyl, and methoxy,
R³¹, R³⁴, R³⁵, R³⁸, R⁴¹, and R⁴² are hydrogen, and R⁵⁰, R⁵¹ are either hydrogen or methyl;
a compound of formula II: 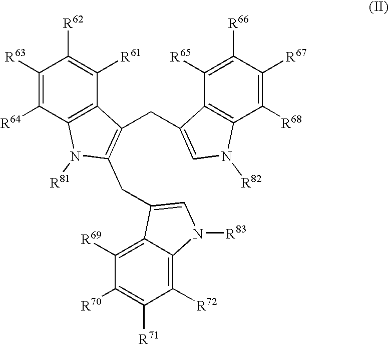
wherein R⁶², R⁶³, R⁶⁶, R⁶⁷, R⁷⁰, and R⁷¹ are substituents independently selected from the group consisting of hydrogen, hydroxyl, and methoxy, and
R⁶¹, R⁶⁴, R⁶⁵, R⁶⁸, R⁶⁹, R⁷², R⁸¹, R⁸² and R⁸³ are hydrogen;
a compound of formula (III): 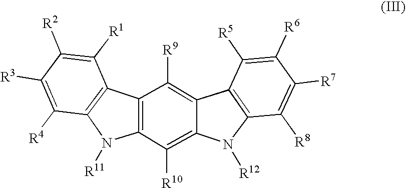
wherein R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, and
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
with the provisos that at least one of R¹, R², R³, R⁴, R⁵, R⁶, R⁷, R⁸, R⁹, R¹⁰, R¹¹ and R¹² is other than hydrogen, and when R¹, R², R³, R⁴, R⁵, R⁶, R⁷, and R⁸ are selected from hydrogen, halo, alkyl and alkoxy, then R¹¹ and R¹² are other than hydrogen and alkyl;
a compound of formula (IV): 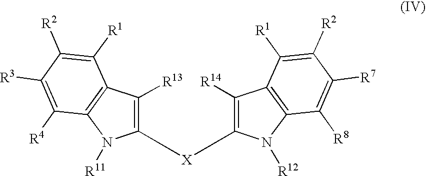
wherein R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
R¹³ and R¹⁴ are defined as for R¹, R², R³, R⁴, R⁵, R⁶, R⁷, and R⁸, with the proviso that at least one of R¹³ and R¹⁴ is other than hydrogen, and
X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹²; and
a compound of formula (V): 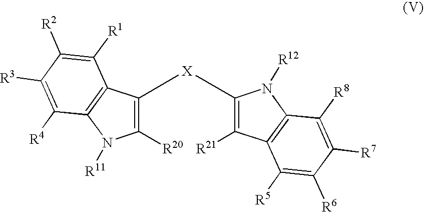
wherein R¹, R², R³, R⁴, R⁵, R⁶, R⁷, R⁸, R¹¹, R¹², and X are defined as for compounds of formula (III), and
R²⁰ and R²¹ are defined as for R¹, R², R³, R⁴, R⁵, R⁶, R⁷, and R⁸.

53. The pharmaceutical composition of claim 51, wherein the one or more DIM-related indoles are selected from the group consisting of diindolylmethane, hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

54. The pharmaceutical composition of claim 51, wherein the composition is formulated for oral administration, vaginal administration, rectal administration or topical administration.
